SEQUENCE LISTING (46 sequences)

SEQ ID NO: 1 - protein, 62 aa, synthetic construct
ILGMTCQSCVKSIEDRISNLKGIISMKVSLEQGSATVKYVPSVVCLQQVCHQIGDMGFEASI

SEQ ID NO: 2 - protein, 67 aa, synthetic construct
VVKLRVEGMTCQSCVSSIEGKVRKLQGVVRVKVSLSNQEAVITYQPYLIQPEDLRDHVNDMGFEAAI

SEQ ID NO: 3 - protein, 70 aa, synthetic construct
TLQLRIDGMHCKSCVLNIEENIGQLLGVQSIQVSLENKTAQVKYDPSCTSPVALQRAIEALPPGNFKVSL

SEQ ID NO: 4 - protein, 67 aa, synthetic construct
TTLIAIAGMTCASCVHSIEGMISQLEGVQQISVSLAEGTATVLYNPSVISPEELRAAIEDMGFEASV

SEQ ID NO: 5 - protein, 67 aa, synthetic construct
KCFLQIKGMTCASCVSNIERNLQKEAGVLSVLVALMAGKAEIKYDPEVIQPLEIAQFIQDLGFEAAV

SEQ ID NO: 6 - protein, 67 aa, synthetic construct
NIELTITGMTCASCVHNIESKLTRTNGITYASVALATSKALVKFDPEIIGPRDIIKIIEEIGFHASL

SEQ ID NO: 7 - protein, 63 aa, synthetic construct
MPEQERQITAREGASRKILSKLSLPTRAWEPAMKKSFAFDNVGYEGGLDGLGPSSQVATSTVR

SEQ ID NO: 8 - protein, 834 aa, synthetic construct
AQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 9 - DNA, 189 bp, synthetic construct
  source 1..189: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcagg

SEQ ID NO: 10 - DNA, 186 bp, synthetic construct
  source 1..186: mol_type = other DNA
atcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcatt

SEQ ID NO: 11 - DNA, 201 bp, synthetic construct
  source 1..201: mol_type = other DNA
gtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatc

SEQ ID NO: 12 - DNA, 210 bp, synthetic construct
  source 1..210: mol_type = other DNA
accctccaactgagaatagatggaatgcattgtaagtcttgcgtcttgaatattgaagaaaatattggccagctcctaggggttcaaagtattcaagtgtccttggagaacaaaactgcccaagtaaagtatgacccttcttgtaccagcccagtggctctgcagagggctatcgaggcacttccacctgggaattttaaagtttctctt

SEQ ID NO: 13 - DNA, 201 bp, synthetic construct
  source 1..201: mol_type = other DNA
accactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtc

SEQ ID NO: 14 - DNA, 201 bp, synthetic construct
  source 1..201: mol_type = other DNA
aagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtc

SEQ ID NO: 15 - DNA, 201 bp, synthetic construct
  source 1..201: mol_type = other DNA
aacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctg

SEQ ID NO: 16 - DNA, 2502 bp, synthetic construct
  source 1..2502: mol_type = other DNA
gcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatc

SEQ ID NO: 17 - DNA, 3267 bp, synthetic construct
  source 1..3267: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcgcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 18 - protein, 1088 aa, synthetic construct
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 19 - DNA, 3108 bp, synthetic construct
  source 1..3108: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 20 - protein, 1035 aa, synthetic construct
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 21 - DNA, 3495 bp, synthetic construct
  source 1..3495: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 22 - protein, 1164 aa, synthetic construct
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 23 - DNA, 3282 bp, synthetic construct
  source 1..3282: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcagggtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcgcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 24 - protein, 1093 aa, synthetic construct
MPEQERQITAREGASRKILSKLSLPTRAWEPAMKKSFAFDNVGYEGGLDGLGPSSQVATSTVRVVKLRVEGMTCQSCVSSIEGKVRKLQGVVRVKVSLSNQEAVITYQPYLIQPEDLRDHVNDMGFEAAIVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 25 - DNA, 3123 bp, synthetic construct
  source 1..3123: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcagggtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 26 - protein, 1040 aa, synthetic construct
MPEQERQITAREGASRKILSKLSLPTRAWEPAMKKSFAFDNVGYEGGLDGLGPSSQVATSTVRVVKLRVEGMTCQSCVSSIEGKVRKLQGVVRVKVSLSNQEAVITYQPYLIQPEDLRDHVNDMGFEAAIMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 27 - DNA, 3510 bp, synthetic construct
  source 1..3510: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcagggtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 28 - protein, 1169 aa, synthetic construct
MPEQERQITAREGASRKILSKLSLPTRAWEPAMKKSFAFDNVGYEGGLDGLGPSSQVATSTVRVVKLRVEGMTCQSCVSSIEGKVRKLQGVVRVKVSLSNQEAVITYQPYLIQPEDLRDHVNDMGFEAAIVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 29 - DNA, 3522 bp, synthetic construct
  source 1..3522: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattgcagaaggaaaggcagcctcctggccctcaaggtccttgcctgcccaggaggctgtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcgcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 30 - protein, 1173 aa, synthetic construct
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIAEGKAASWPSRSLPAQEAVVKLRVEGMTCQSCVSSIEGKVRKLQGVVRVKVSLSNQEAVITYQPYLIQPEDLRDHVNDMGFEAAIVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 31 - DNA, 3363 bp, synthetic construct
  source 1..3363: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattgcagaaggaaaggcagcctcctggccctcaaggtccttgcctgcccaggaggctgtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 32 - protein, 1120 aa, synthetic construct
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIAEGKAASWPSRSLPAQEAVVKLRVEGMTCQSCVSSIEGKVRKLQGVVRVKVSLSNQEAVITYQPYLIQPEDLRDHVNDMGFEAAIMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 33 - DNA, 3750 bp, synthetic construct
  source 1..3750: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattgcagaaggaaaggcagcctcctggccctcaaggtccttgcctgcccaggaggctgtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 34 - protein, 1249 aa, synthetic construct
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIAEGKAASWPSRSLPAQEAVVKLRVEGMTCQSCVSSIEGKVRKLQGVVRVKVSLSNQEAVITYQPYLIQPEDLRDHVNDMGFEAAIVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 35 - DNA, 4398 bp, synthetic construct
  source 1..4398: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattgcagaaggaaaggcagcctcctggccctcaaggtccttgcctgcccaggaggctgtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcaagagcaaagtggctcccttaagcctgggaccaattgatattgagcggttacaaagcactaacccaaagagacctttatcttctgctaaccagaattttaataattctgagaccttggggcaccaaggaagccatgtggtcaccctccaactgagaatagatggaatgcattgtaagtcttgcgtcttgaatattgaagaaaatattggccagctcctaggggttcaaagtattcaagtgtccttggagaacaaaactgcccaagtaaagtatgacccttcttgtaccagcccagtggctctgcagagggctatcgaggcacttccacctgggaattttaaagtttctcttcctgatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 36 - protein, 1465 aa, synthetic construct
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIAEGKAASWPSRSLPAQEAVVKLRVEGMTCQSCVSSIEGKVRKLQGVVRVKVSLSNQEAVITYQPYLIQPEDLRDHVNDMGFEAAIKSKVAPLSLGPIDIERLQSTNPKRPLSSANQNFNNSETLGHQGSHVVTLQLRIDGMHCKSCVLNIEENIGQLLGVQSIQVSLENKTAQVKYDPSCTSPVALQRAIEALPPGNFKVSLPDGAEGSGTDHRSSSSHSPGSPPRNQVQGTCSTTLIAIAGMTCASCVHSIEGMISQLEGVQQISVSLAEGTATVLYNPSVISPEELRAAIEDMGFEASVVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 37 - protein, 18 aa, synthetic construct
AEGKAASWPSRSLPAQEA

SEQ ID NO: 38 - protein, 47 aa, synthetic construct
KSKVAPLSLGPIDIERLQSTNPKRPLSSANQNFNNSETLGHQGSHVV

SEQ ID NO: 39 - protein, 32 aa, synthetic construct
PDGAEGSGTDHRSSSSHSPGSPPRNQVQGTCS

SEQ ID NO: 40 - protein, 62 aa, synthetic construct
VSESCSTNPLGNHSAGNSMVQTTDGTPTSVQEVAPHTGRLPANHAPDILAKSPQSTRAVAPQ

SEQ ID NO: 41 - protein, 9 aa, synthetic construct
MEDYAGSDG

SEQ ID NO: 42 - DNA, 54 bp, synthetic construct
  source 1..54: mol_type = other DNA
gcagaaggaaaggcagcctcctggccctcaaggtccttgcctgcccaggaggct

SEQ ID NO: 43 - DNA, 141 bp, synthetic construct
  source 1..141: mol_type = other DNA
aagagcaaagtggctcccttaagcctgggaccaattgatattgagcggttacaaagcactaacccaaagagacctttatcttctgctaaccagaattttaataattctgagaccttggggcaccaaggaagccatgtggtc

SEQ ID NO: 44 - DNA, 96 bp, synthetic construct
  source 1..96: mol_type = other DNA
cctgatggagccgaagggagtgggacagatcacaggtcttccagttctcattcccctggctccccaccgagaaaccaggtccagggcacatgcagt

SEQ ID NO: 45 - DNA, 186 bp, synthetic construct
  source 1..186: mol_type = other DNA
gtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcag

SEQ ID NO: 46 - DNA, 27 bp, synthetic construct
  source 1..27: mol_type = other DNA
atggaggactacgcaggctccgatggc

(19) United States
(12) Patent Application Publication
(10) Pub. No.: US 2026/0273099 A1
(43) Pub. Date: Sep. 17, 2026

(54) A TRUNCATED ATP7B AND THE USE THEREOF

(71) Applicant: LINGYI BIOTECH CO., LTD., Xiaoshan Dist Hangzhou, Zhejiang (CN)

(72) Inventors: Qing LIN, Shanghai (CN); Yixiong CHEN, Shanghai (CN); Yuzhe PENG, Shanghai (CN); Qiang WU, Shanghai (CN)

(73) Assignee: LINGYI BIOTECH CO., LTD., Xiaoshan Dist Hangzhou, Zhejiang (CN)

(21) Appl. No.: 19/162,336

(22) PCT Filed: Mar. 6, 2024
(86) PCT No.: PCT/CN2024/080394

§ 371 Date: Sep. 4, 2025

(30) Foreign Application Priority Data
Mar. 6, 2023 (WO) PCT/CN2023/079933

(51) Int. Cl. A61K 48/00; A61K 38/00; A61P 3/00; C12N 9/00; C12N 15/86

(52) U.S. Cl. CPC A61K 48/005; A61P 3/00; C12N 9/00; C12N 15/86; A61K 38/00; C07K 2319/00; C12N 2750/14143; C12N 2830/42; C12N 2830/50

(57) ABSTRACT

This disclosure relates to a truncated ATP7B in which CRE5 and/or CRE6 are present, optionally, CRE1 and/or CRE2 are present. The disclosure also provides an adeno-associated viral vector encoding a the truncated ATP7B for use in gene therapy for treating Wilson's disease (WD).

### PRIORITY

This application claims the benefit of, and priority to, PCT Application No. PCT/CN2023/079933, filed Mar. 6, 2023, the entire contents of which are hereby incorporated by reference.

### TECHNICAL FIELD OF THE INVENTION

This disclosure relates generally to a truncated ATPase copper transporting beta (ATP7B) and its nucleic acid sequence, recombinant nucleic acid construct, adeno-associated viral vectors, and methods of their use in gene therapy for treating Wilson's disease (WD).

### BACKGROUND OF THE INVENTION

Wilson's disease (WD) is an autosomal recessively genetic disorder of copper metabolism, which primarily results in copper accumulation in liver and subsequently in the neurological system and others. WD is a rare disease with an average worldwide prevalence of 1:30,000, caused by mutations of the ATP7B gene encoding for a P-type copper transporting ATPase, which is located on chromosome 13.

ATP7B is expressed mainly in hepatocytes and functions as the transmembrane transporter of copper. Absent or reduced function of ATP7B leads to decreased hepatocellular excretion of copper into bile, and causes copper accumulation in the liver and subsequent organs. Without normal copper metabolism, ceruloplasmin lacks copper conjugation, resulting in additional hemolytic anemia. ATP7B has a basic P-type ATPase architecture with a cytosolic region composed of phosphorylation (P-), ATP-binding (N-) and actuator/dephosphorylation (A-) domains and a membrane part where eight transmembrane helices (transmembrane domains, TMDs) form an intramembranous copper channel. A unique structural feature of this multi-domain protein, is the presence of a large, cytosolic N-terminal tail containing six approximate 70 amino acids long independently folded copper (II) responsive elements (CREs). CREs were named from the N-terminal, with CRE1 being the first domain from the N-terminus and CRE6 being the last domain closest to the membrane-spanning part of ATP7B.

### SUMMARY OF THE INVENTION

This disclosure provides a truncated ATP7B comprising one or more sequences selected from CRE5, CRE6, and its variants thereof, and one or more sequences selected from CRE1, CRE2, and its variants thereof.

In some embodiment, the truncated ATP7B further comprises a flexible linker connecting the sequences to each other.

In some embodiment, the truncated ATP7B has a structure as shown in Formula Ia from N-terminus to C-terminus:

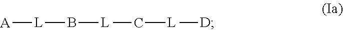

wherein A, B, C, or D is selected from CRE1, CRE2, CRE5, CRE6 and variants thereof; A, B, C, and D are different, respectively; A and B are present, C and/or D are optionally present; and
L is none or a flexible linker.

In some embodiment, this disclosure provides a truncated ATP7B in which 1) CRE5 or variant thereof and/or 2) CRE6 or variant thereof is present; and 3) CRE1 or variant thereof and/or 4) CRE2 or variant thereof is present.

Wild type ATP7B is composed of several independently folded Cu (II) responsive elements (CREs) between the leader peptide (LP) and C-terminal region (CTR). Six CREs could be identified from the N-terminal to the membrane-spanning part of ATP7B. CREs constituting truncated ATP7B could be arranged in any order, and combined directly without any sequences, or with any flexible sequences including endogenous linkers between CREs.

In some embodiment, this disclosure provides a truncated ATP7B in which CRE1 or variant thereof and CRE5 or variant thereof are present.

In some embodiment, this disclosure provides a truncated ATP7B in which CRE1 or variant thereof and CRE6 or variant thereof are present.

In some embodiment, this disclosure provides a truncated ATP7B in which CRE1 or variant thereof, CRE5 or variant thereof, and CRE6 or variant thereof are present.

In some embodiment, this disclosure provides a truncated ATP7B in which CRE2 or variant thereof and CRE5 or variant thereof are present.

In some embodiment, this disclosure provides a truncated ATP7B in which CRE2 or variant thereof and CRE6 or variant thereof are present.

In some embodiment, this disclosure provides a truncated ATP7B in which CRE2 or variant thereof, CRE5 or variant thereof, and CRE6 or variant thereof are present.

In some embodiment, this disclosure provides a truncated ATP7B in which CRE1 or variant thereof, CRE2 or variant thereof, and CRE5 or variant thereof are present.

In some embodiment, this disclosure provides a truncated ATP7B in which CRE1 or variant thereof, CRE2 or variant thereof, and CRE6 or variant thereof are present.

In some embodiment, this disclosure provides a truncated ATP7B in which CRE1 or variant thereof, CRE2 or variant thereof, CRE5 or variant thereof, and CRE6 or variant thereof are present.

In some embodiment, CRE3 is deleted in the truncated ATP7B. Preferably, this disclosure provides a truncated ATP7B in which CRE1 or variant thereof and CRE5 or variant thereof are present, but CRE2, CRE3, CRE4, and CRE6 have been deleted. Preferably, this disclosure provides a truncated ATP7B in which CRE1 or variant thereof, and CRE6 or variant thereof are present, but CRE2, CRE3, CRE4, and CRE5 have been deleted. Preferably, this disclosure provides a truncated ATP7B in which CRE1 or variant thereof, CRE5 or variant thereof, and CRE6 or variant thereof are present, but CRE2, CRE3, and CRE4 have been deleted. Preferably, this disclosure provides a truncated ATP7B in which CRE2 or variant thereof and CRE5 or variant thereof are present, but CRE1, CRE3, CRE4, and CRE6 have been deleted. Preferably, this disclosure provides a truncated ATP7B in which CRE2 or variant thereof and CRE6 or variant thereof are present, but CRE1, CRE3, CRE4 and CRE5 have been deleted. Preferably, this disclosure provides a truncated ATP7B in which CRE2 or variant thereof, CRE5 or variant thereof, and CRE6 or variant thereof are present, but CRE1, CRE3, and CRE4 have been deleted. Preferably, this disclosure provides a truncated ATP7B in which CRE1 or variant thereof, CRE2 or variant thereof, and CRE5 or variant thereof are present, but CRE3, CRE4, and CRE6 have been deleted. Preferably, this disclosure provides a truncated ATP7B in which CRE1 or variant thereof, CRE2 or variant thereof, and CRE6 or variant thereof are present, but CRE3, CRE4 and CRE5 have been deleted. Preferably, this disclosure provides a truncated ATP7B in which CRE1 or variant thereof, CRE2 or variant thereof, CRE5 or variant thereof, and CRE6 or variant thereof are present, but CRE3, and CRE4 have been deleted.

In some embodiment, the truncated ATP7B comprises an amino acid sequence selected from the group consisting of SEQ ID NOs: 18, 20, 22, 24, 26, 28, 30, 32, and 34, and at least 80%, at least 85%, at least 90%, at least 95%, at least 99%, at least 99.5%, at least 99.8% identical to SEQ ID NOs: 18, 20, 22, 24, 26, 28, 30, 32, and 34, wherein the truncated ATP7B retains the functionality of transmembrane copper transportation.

In some embodiment, CRE3 is deleted.

In some embodiment, CRE3 is not deleted in the truncated ATP7B. Preferably, this disclosure provides a truncated ATP7B in which CRE1 or variant thereof, CRE3 or variant thereof, and CRE5 or variant thereof are present, but CRE2, CRE4 and CRE6 have been deleted. Preferably, this disclosure provides a truncated ATP7B in which CRE1 or variant thereof, CRE3 or variant thereof, and CRE6 or variant thereof are present, but CRE2, CRE4, and CRE5 have been deleted. Preferably, this disclosure provides a truncated ATP7B in which CRE1 or variant thereof, CRE3 or variant thereof, CRE5 or variant thereof, and CRE6 or variant thereof are present, but CRE2 and CRE4 have been deleted. Preferably, this disclosure provides a truncated ATP7B in which CRE2 or variant thereof, CRE3 or variant thereof, and CRE5 or variant thereof are present, but CRE1, CRE4 and CRE6 have been deleted. Preferably, this disclosure provides a truncated ATP7B in which CRE2 or variant thereof, CRE3 or variant thereof, and CRE6 or variant thereof are present, but CRE1, CRE4, and CRE5 have been deleted. Preferably, this disclosure provides a truncated ATP7B in which CRE2 or variant thereof, CRE3 or variant thereof, CRE5 or variant thereof, and CRE6 or variant thereof are present, but CRE1 and CRE4 have been deleted. Preferably, this disclosure provides a truncated ATP7B in which CRE1 or variant thereof, CRE2 or variant thereof, CRE3 or variant thereof, and CRE5 or variant thereof are present, but CRE4, and CRE6 have been deleted. Preferably, this disclosure provides a truncated ATP7B in which CRE1 or variant thereof, CRE2 or variant thereof, CRE3 or variant thereof, and CRE6 or variant thereof are present, but CRE4 and CRE5 have been deleted. Preferably, this disclosure provides a truncated ATP7B in which CRE1 or variant thereof, CRE2 or variant thereof, CRE3 or variant thereof, CRE5 or variant thereof, and CRE6 or variant thereof are present, but CRE4 have been deleted.

In some embodiment, CRE3 has been deleted, but CRE5 or variant thereof and/or CRE6 or variant thereof is present, CRE1 or variant thereof and/or CRE2 or variant thereof is present, the said truncated ATP7B further comprises CRE4 or variant thereof. Preferably, this disclosure provides a truncated ATP7B in which CRE1 or variant thereof, CRE4 or variant thereof, and CRE5 or variant thereof are present, but CRE2, CRE3 and CRE6 have been deleted. Preferably, this disclosure provides a truncated ATP7B in which CRE1 or variant thereof, CRE4 or variant thereof, and CRE6 or variant thereof are present, but CRE2, CRE3, CRE5 have been deleted. Preferably, this disclosure provides a truncated ATP7B in which CRE1 or variant thereof, CRE4 or variant thereof, CRE5 or variant thereof, and CRE6 or variant thereof are present, but CRE2 and CRE3 have been deleted. Preferably, this disclosure provides a truncated ATP7B in which CRE2 or variant thereof, CRE4 or variant thereof, and CRE5 or variant thereof are present, but CRE1, CRE3 and CRE6 have been deleted. Preferably, this disclosure provides a truncated ATP7B in which CRE2 or variant thereof, CRE4 or variant thereof, and CRE6 or variant thereof are present, but CRE1, CRE3, and CRE5 have been deleted. Preferably, this disclosure provides a truncated ATP7B in which CRE2 or variant thereof, CRE4 or variant thereof, CRE5 or variant thereof, and CRE6 or variant thereof are present, but CRE1 and CRE3 have been deleted. Preferably, this disclosure provides a truncated ATP7B in which CRE1 or variant thereof, CRE2 or variant thereof, CRE4 or variant thereof, and CRE5 or variant thereof are present, but CRE3, and CRE6 have been deleted. Preferably, this disclosure provides a truncated ATP7B in which CRE1 or variant thereof, CRE2 or variant thereof, CRE4 or variant thereof, and CRE6 or variant thereof are present, but CRE3 and CRE5 have been deleted. Preferably, this disclosure provides a truncated ATP7B in which CRE1 or variant thereof, CRE2 or variant thereof, CRE4 or variant thereof, CRE5 or variant thereof, and CRE6 or variant thereof are present, but CRE3 have been deleted.

In another aspect, this disclosure provides truncated ATP7B comprises CRE5 or variant thereof, and further comprises CRE3 or variant thereof or CRE4 or variant thereof.

In another aspect, this disclosure provides a nucleic acid sequence encoding the said truncated ATP7B.

In some embodiment, truncated ATP7B nucleotide comprises a sequence selected from the group consisting of SEQ ID NOs: 17, 19, 21, 23, 25, 27, 29, 31, and 33, and at least 80%, at least 85%, at least 90%, at least 95%, at least 99%, at least 99.5%, at least 99.8% identical to SEQ ID NOs: 17, 19, 21, 23, 25, 27, 29, 31, and 33, wherein the truncated ATP7B nucleotide encodes the truncated ATP7B protein that retains the functionality of transmembrane copper transportation.

In another aspect, this disclosure provides RNA polynucleotide encoding the truncated ATP7B in present disclosure. The RNA comprises a messenger RNA (mRNA) or a circular RNA (circRNA).

Messenger RNA (mRNA) is any ribonucleic acid that encodes a (at least one) protein (a naturally-occurring, non-naturally-occurring, or modified polymer of amino acids) and can be translated to produce the encoded protein in vitro, in vivo, or in situ. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence identical to the DNA, where each “T” of the DNA sequence is substituted with “U.” Likewise, any of the RNAs disclosed and identified by a particular sequence identification number herein also disclose the corresponding DNA sequence identical to the RNA, where each “U” of the RNA sequence is substituted with “T.”

In some embodiments, the RNA comprises at least one modified nucleotide. The terms “modification” or, as appropriate, “modified” refer to modification with respect to A, G, U or C ribonucleotides. Generally, herein, these terms are not intended to refer to the ribonucleotide modifications in naturally occurring 5′-terminal mRNA cap moieties. In a polypeptide, the term “modification” refers to a modification as compared to the canonical set of 20 amino acids.

In another aspect, this disclosure provides a recombinant nucleic acid construct comprising:
(a) a 5′-inverted terminal repeat (ITR) sequence;
(b) a promoter sequence;
(c) the said nucleic acid sequence encoding the said truncated ATP7B;
(d) a poly(A) sequence; and
(e) a 3′-ITR sequence.

In some embodiment, the recombinant nucleic acid construct further comprises one or more enhancer sequences.

In some embodiment, the recombinant nucleic acid construct further comprises one or more intron sequences.

In some embodiment, the recombinant nucleic acid construct further comprises one or more signal sequences.

In another aspect, this disclosure provides a recombinant vector comprising the said recombinant nucleic acid construct.

In some embodiment, the vector is a viral vector, preferably is an AAV vector.

In another aspect, this disclosure provides the vector of present disclosure and capsid protein.

In some embodiment, the AAV capsid is selected from the group consisting of: serotype 9, 8, 1, 2, 3, 3B, 4, 5, 6, 7, 10, 11, 12, 13, rh10, or hu37 as well as any one of the AAV serotype isolated from human and nonhuman mammalians or variant thereof.

In another aspect, this disclosure provides a pharmaceutical composition comprising the said truncated ATP7B, the said nucleic acid sequence, the said recombinant nucleic construct, the said recombinant vector, or the said rAAV.

In another aspect, this disclosure provides a method for treating a disease in the subjects, comprising administrating the effect amount of the said truncated ATP7B, the said nucleic acid sequence, the said recombinant nucleic construct, the said recombinant vector, the said rAAV, or the said pharmaceutical composition.

In some embodiment, the disease is an ATP7B related disease, preferably is Wilson's disease.

In some embodiment, the subject is mammalian, preferably human.

### BRIEF DESCRIPTION OF THE DRAWINGS

The invention can be more completely understood with reference to the following drawings.

FIG. 1 showed the relative copper transportation capacity of truncated ATP7B forms with single CRE evaluated by Dual Luciferase Assay. Higher relative Fluci/Rluci ratio represented lower copper transportation capacity. CRE5 exhibited the highest copper transportation capacity among six CREs. ***: p<0.001. Plot represented mean±SEM, while n=3 in each group.

FIG. 2 showed the combination of CRE5 significantly elevated copper transportation capacity of CRE1, CRE2, CRE3 and CRE4. ***: p<0.001. Plot represented mean±SEM, while n=3 in each group.

FIG. 3 showed the combination of CRE1 significantly elevated copper transportation capacity of CRE5, CRE6, CRE5+CRE6, and CRE4+CRE6. *: p<0.05, ***: p<0.001. Plot represented mean±SEM, while n=15 in each group.

FIG. 4 showed the combination of CRE2 significantly elevated copper transportation capacity of CRE5, CRE6, CRE5+CRE6, and CRE4+CRE6. **: 0.001<p<0.01, ***: p<0.001. Plot represented mean±SEM, while n=15 in each group.

FIG. 5 showed the combination of CRE3 had no further enhancement effect of copper transportation capacity on CRE6, CRE5+CRE6, CRE1+CRE5, and CRE1+CRE6, among which some combinations even had negative effect of copper transportation capacity. NS: p>0.05. Plot represented mean±SEM, while n=15 in each group.

FIG. 6 showed although the combination of CRE3 or CRE4 had negative effect of on some CRE combinations, adding CRE3 or CRE4 on these CRE combinations yet overweighed or were comparable to CRE5+CRE6 on the copper transportation capacity. NS: p>0.05. **: 0.001<p<0.01, ***: p<0.001. Plot represented mean±SEM, while n=15 in each group.

FIG. 7 showed the combination of CRE1+CRE2 significantly elevated copper transportation capacity of CRE5, CRE6, and CRE5+CRE6. ***: p<0.001. Plot represented mean±SEM, while n=15 in each group.

FIG. 8 showed the specific CRE combinations exhibited comparative copper transportation capacity to wild type form. Plot represented mean±SEM, while n=15 in each group.

FIG. 9 showed the truncated ATP7B with specific combinations of CRE or CRE-schemes could be efficiently packaged in AAV8 with the production yield>2.0×10¹¹ vg/mL, which is much higher than the AAV8 encoding wild type full length ATP7B.

FIG. 10 showed the packaged AAV8 of the truncated ATP7B with specific combinations of CREs or CRE-schemes could reverse ceruloplasmin (Cp) activity (a) and tissue copper accumulation (b) in Wilson's Disease mouse model. *: p<0.05, **: p<0.01. Plot represented mean±SEM, while n=3 in each group.

### DETAILED DESCRIPTION OF THE INVENTION

### Truncated a TP7B

Wild type ATP7B is composed of several independently folded copper (II) responsive elements (CREs) between the leader peptide (LP) and C-terminal region (CTR). Six CREs could be identified from the N-terminal to the membrane-spanning region of ATP7B. The sequences of CREs are shown as SEQ ID NO:1-6. Truncated ATP7B nucleotide described herein comprises a nucleotide sequence encoding a functional fragment or domain of ATP7B. Truncated ATP7B comprises a deletion of one or more CREs. Truncated ATP7B can also be a recombinant polypeptide comprising one or more CREs. CREs constituting truncated ATP7B could be arranged in any order, and combined directly without any insertions, or with any flexible sequences including endogenous linkers between CREs.

This disclosure provides a truncated ATP7B in which CRE5 or variant thereof and/or CRE6 or variant thereof is present, and CRE1 or variant thereof and/or CRE2 or variant thereof is present.

The term “variant” comprises an amino acid sequence which differs from that of a native sequence by virtue of at least one “amino acid modification” as herein defined. Preferably, the variant has at least one amino acid substitution, or deletion compared to a native sequence. The variant herein will preferably possess at least about 80% homology with a native sequence, and most preferably at least about 90% homology therewith, more preferably at least about 95% homology therewith, and retains the functionality of native sequence.

In some embodiment, this disclosure provides a truncated ATP7B in which CRE1 or variant thereof and CRE5 or variant thereof are present, but CRE2, CRE3, CRE4 and CRE6 have been deleted. In some embodiment, this disclosure provides a truncated ATP7B comprising amino acids sequences of SEQ ID NO: 1 and SEQ ID NO: 5. The truncated ATP7B further comprises a leader peptide (LP) and a C-terminal region (CTR). In some embodiment, this disclosure provides a truncated ATP7B comprising an amino acid sequence of SEQ ID NO: 18 or an amino acid sequence at least 80%, 85%, 90%, 91%, 92%, 93%, 94%, 95%, 96%, 97%, 98%, 99%, 99.5% identical to SEQ ID NO: 18, and the truncated ATP7B retains the functionality of transmembrane copper transportation.

In some embodiment, this disclosure provides a truncated ATP7B in which CRE1 or variant thereof and CRE6 or variant thereof are present, but CRE2, CRE3, CRE4 and CRE5 have been deleted. In some embodiment, this disclosure provides a truncated ATP7B comprising amino acids sequences of SEQ ID NO: 1 and SEQ ID NO: 6. The truncated ATP7B further comprises a leader peptide (LP) and a C-terminal region (CTR). In some embodiment, this disclosure provides a truncated ATP7B comprising an amino acid sequence of SEQ ID NO: 20 or an amino acid sequence at least 80%, 85%, 90%, 91%, 92%, 93%, 94%, 95%, 96%, 97%, 98%, 99%, 99.5% identical to SEQ ID NO: 20, and the truncated ATP7B retains the functionality of transmembrane copper transportation.

In some embodiment, this disclosure provides a truncated ATP7B in which CRE1 or variant thereof, CRE5 or variant thereof, and CRE6 or variant thereof are present, but CRE2, CRE3, and CRE4 have been deleted. In some embodiment, this disclosure provides a truncated ATP7B comprising amino acids sequences of SEQ ID NO: 1, SEQ ID NO: 5, and SEQ ID NO: 6. The truncated ATP7B further comprises a leader peptide (LP) and a C-terminal region (CTR). In some embodiment, this disclosure provides a truncated ATP7B comprising an amino acid sequence of SEQ ID NO: 22 or an amino acid sequence at least 80%, 85%, 90%, 91%, 92%, 93%, 94%, 95%, 96%, 97%, 98%, 99%, 99.5% identical to SEQ ID NO: 22, and the truncated ATP7B retains the functionality of transmembrane copper transportation.

In some embodiment, this disclosure provides a truncated ATP7B in which CRE2 or variant thereof and CRE5 or variant thereof are present, but CRE1, CRE3, CRE4 and CRE6 have been deleted. In some embodiment, this disclosure provides a truncated ATP7B comprising amino acids sequences of SEQ ID NO: 2, and SEQ ID NO: 5. The truncated ATP7B further comprises a leader peptide (LP) and a C-terminal region (CTR). In some embodiment, this disclosure provides a truncated ATP7B comprising an amino acid sequence of SEQ ID NO: 24 or an amino acid sequence at least 80%, 85%, 90%, 91%, 92%, 93%, 94%, 95%, 96%, 97%, 98%, 99%, 99.5% identical to SEQ ID NO: 24, and the truncated ATP7B retains the functionality of transmembrane copper transportation.

In some embodiment, this disclosure provides a truncated ATP7B in which CRE2 or variant thereof and CRE6 or variant thereof are present, but CRE1, CRE3, CRE4 and CRE5 have been deleted. In some embodiment, this disclosure provides a truncated ATP7B comprising amino acids sequences of SEQ ID NO: 2, and SEQ ID NO: 6. The truncated ATP7B further comprises a leader peptide (LP) and a C-terminal region (CTR). In some embodiment, this disclosure provides a truncated ATP7B comprising an amino acid sequence of SEQ ID NO: 26 or an amino acid sequence at least 80%, 85%, 90%, 91%, 92%, 93%, 94%, 95%, 96%, 97%, 98%, 99%, 99.5% identical to SEQ ID NO: 26, and the truncated ATP7B retains the functionality of transmembrane copper transportation.

In some embodiment, this disclosure provides a truncated ATP7B in which CRE2 or variant thereof, CRE5 or variant thereof, and CRE6 or variant thereof are present, but CRE1, CRE3, and CRE4 have been deleted. In some embodiment, this disclosure provides a truncated ATP7B comprising amino acids sequences of SEQ ID NO: 2, SEQ ID NO: 5, and SEQ ID NO: 6. The truncated ATP7B further comprises a leader peptide (LP) and a C-terminal region (CTR). In some embodiment, this disclosure provides a truncated ATP7B comprising an amino acid sequence of SEQ ID NO: 28 or an amino acid sequence at least 80%, 85%, 90%, 91%, 92%, 93%, 94%, 95%, 96%, 97%, 98%, 99%, 99.5% identical to SEQ ID NO: 28, and the truncated ATP7B retains the functionality of transmembrane copper transportation.

In some embodiment, this disclosure provides a truncated ATP7B in which CRE1 or variant thereof, CRE2 or variant thereof, and CRE5 or variant thereof are present, but CRE3, CRE4, and CRE6 have been deleted. In some embodiment, this disclosure provides a truncated ATP7B comprising amino acids sequences of SEQ ID NO: 1, SEQ ID NO: 2, and SEQ ID NO: 5. The truncated ATP7B further comprises a leader peptide (LP) and a C-terminal region (CTR). In some embodiment, this disclosure provides a truncated ATP7B comprising an amino acid sequence of SEQ ID NO: 30 or an amino acid sequence at least 80%, 85%, 90%, 91%, 92%, 93%, 94%, 95%, 96%, 97%, 98%, 99%, 99.5% identical to SEQ ID NO: 30, and the truncated ATP7B retains the functionality of transmembrane copper transportation.

In some embodiment, this disclosure provides a truncated ATP7B in which CRE1 or variant thereof, CRE2 or variant thereof, and CRE6 or variant thereof are present, but CRE3, CRE4 and CRE5 have been deleted. In some embodiment, this disclosure provides a truncated ATP7B comprising amino acids sequences of SEQ ID NO: 1, SEQ ID NO: 2, and SEQ ID NO: 6. The truncated ATP7B further comprises a leader peptide (LP) and a C-terminal region (CTR). In some embodiment, this disclosure provides a truncated ATP7B comprising an amino acid sequence of SEQ ID NO: 32 or an amino acid sequence at least 80%, 85%, 90%, 91%, 92%, 93%, 94%, 95%, 96%, 97%, 98%, 99%, 99.5% identical to SEQ ID NO: 32, and the truncated ATP7B retains the functionality of transmembrane copper transportation.

In some embodiment, this disclosure provides a truncated ATP7B in which CRE1 or variant thereof, CRE2 or variant thereof, CRE5 or variant thereof, and CRE6 or variant thereof are present, but CRE3, and CRE4 have been deleted. In some embodiment, this disclosure provides a truncated ATP7D comprising amino acids sequences of SEQ ID NO: 1, SEQ ID NO: 2, i0 SEQ ID NO: 5, and SEQ ID NO: 6. The truncated ATP7B further comprises a leader peptide (LP) and a C-terminal region (CTR). In some embodiment, this disclosure provides a truncated ATP7B comprising an amino acid sequence of SEQ ID NO: 34 or an amino acid sequence at least 80%, 85%, 90%, 91%, 92%, 93%, 94%, 95%, 96%, 97%, 98%, 99%, 99.5% identical to SEQ ID NO: 34, and the truncated ATP7B retains the functionality of transmembrane copper transportation.

TABLE 1
Sequences of the complete and truncated ATP7B

| | | SEQ | | SEQ |
|---|---|---|---|---|
| | | ID | | ID |
| | Nucleotide sequence | NO | Amino acid sequence | NO |
| Complete | atgcctgagcaggagagacagatcacagccagagaaggg | 35 | MPEQERQITAREGASRKI | 36 |
| ATP7B | gccagtcggaaaatcttatctaagctttctttgcctacccgtgcc | | LSKLSLPTRAWEPAMKK | |
| | tgggaaccagcaatgaagaagagttttgcttttgacaatgttg | | SFAFDNVGYEGGLDGLG | |
| | gctatgaaggtggtctggatggcctgggcccttcttctcaggt | | PSSQVATSTVRILGMTC | |
| | ggccaccagcacagtcaggatcttgggcatgacttgccagtc | | QSCVKSIEDRISNLKGIIS | |
| | atgtgtgaagtccattgaggacaggatttccaatttgaaaggc | | MKVSLEQGSATVKYVPS | |
| | atcatcagcatgaaggtttccctggaacaaggcagtgccact | | VVCLQQVCHQIGDMGF | |
| | gtgaaatatgtgccatcggttgtgtgcctgcaacaggtttgcc | | EASIAEGKAASWPSRSLP | |
| | atcaaattggggacatgggcttcgaggccagcattgcagaa | | AQEAVVKLRVEGMTCQ | |
| | ggaaaggcagcctcctggccctcaaggtccttgcctgcccag | | SCVSSIEGKVRKLQGVVR | |
| | gaggctgtggtcaagctccgggggagggcatgacctgcca | | VKVSLSNQEAVITYQPYL | |
| | gtcctgtgtcagctccattgaaggcaaggtccggaaactgca | | IQPEDLRDHVNDMGFE | |
| | aggagtagtgagagtcaaagtctcactcagcaaccaagagg | | AAIKSKVAPLSLGPIDIER | |
| | ccgtcatcacttatcagccttatctcattcagcccgaagacctca | | LQSTNPKRPLSSANQNF | |
| | gggaccatgtaaatgacatgggatttgaagctgccatcaaga | | NNSETLGHQGSHVVTL | |
| | gcaaagtggctcccttaagcctgggaccaattgatattgagc | | QLRIDGMHCKSCVLNIE | |
| | ggttacaaagcactaacccaaagagacctttatcttctgctaac | | ENIGQLLGVQSIQVSLE | |
| | cagaattttaataattctgagaccttggggcaccaaggaagcc | | NKTAQVKYDPSCTSPVA | |
| | atgtggtcaccctccaactgagaatagatggaatgcattgtaa | | LQRAIEALPPGNFKVSLP | |
| | gtcttgcgtcttgaatattgaagaaaatattggccagctcctag | | DGAEGSGTDHRSSSSHS | |
| | gggttcaaagtattcaagtgtccttggagaacaaaactgccca | | PGSPPRNQVQGTCSTTL | |
| | agtaaagtatgacccttcttgtaccagcccagtggctctgcag | | IAIAGMTCASCVHSIEG | |
| | agggctatcgaggcacttccacctgggaattttaaagtttctctt | | MISQLEGVQQISVSLAE | |
| | cctgatggagccgaagggagtgggacagatcacaggtcttc | | GTATVLYNPSVISPEELR | |
| | cagttctcattcccctggctccccaccgagaaaccaggtccag | | AAIEDMGFEASVVSESC | |
| | ggcacatgcagtaccactctgattgccattgccggcatgacct | | STNPLGNHSAGNSMVQ | |
| | gtgcatcctgtgtccattccattgaaggcatgatctcccaactg | | TTDGTPTSVQEVAPHTG | |
| | gaaggggtgcagcaaatatcggtgtctttggccgaagggac | | RLPANHAPDILAKSPQS | |
| | tgcaacagttctttataatccctctgtaattagcccagaagaact | | TRAVAPQKCFLQIKGMT | |
| | cagagctgctatagaagacatgggatttgaggcttcagtcgtt | | CASCVSNIERNLQKEAG | |
| | tctgaaagctgttctactaaccctcttggaaaccacagtgctgg | | VLSVLVALMAGKAEIKY | |
| | gaattccatggtgcaaactacagatggtacacctacatctgtg | | DPEVIQPLEIAQFIQDLG | |
| | caggaagtggctccccacactgggaggctccctgcaaaccat | | FEAAVMEDYAGSDGNIE | |
| | gccccggacatcttggcaaagtccccacaatcaaccagagca | | LTITGMTCASCVHNIESK | |
| | gtggcaccgcagaagtgcttcttacagatcaaaggcatgacc | | LTRTNGITYASVALATSK | |
| | tgtgcatcctgtgtgtctaacatagaaaggaatctgcagaaag | | ALVKFDPEIIGPRDIIKII | |
| | aagctggtgttctctccgtgttggttgccttgatggcaggaaa | | EEIGFHASLAQRNPNAHHL | |
| | ggcagagatcaagtatgacccagaggtcatccagcccctcg | | DHKMEIKQWKKSFLCSL | |
| | agatagctcagttcatccaggacctgggttttgaggcagcagt | | VFGIPVMALMIYMLIPSN | |
| | catggaggactacgcaggctccgatggcaacattgagctga | | EPHQSMVLDHNIIPGLSI | |
| | caatcacagggatgacctgcgcgtcctgtgtccacaacatag | | LNLIFFILCTFVQLLGGW | |
| | agtccaaactcacgaggacaaatggcatcacttatgcctccgt | | YFYVQAYKSLRHRSAN | |
| | tgcccttgccaccagcaaagcccttgttaagtttgacccggaa | | MDVLIVLATSIAYVYSLVI | |
| | attatcggtccacgggatattatcaaaattattgaggaaattgg | | LVVAVAEKAERSPVTFFD | |
| | ctttcatgcttccctggcccagagaaaccccaacgctcatcact | | TPPMLFVFIALGRWLEHL | |
| | tggaccacaagatggaaataaagcagtggaagaagtctttcc | | AKSKTSEALAKLMSLQA | |
| | tgtgcagcctggtgtttggcatccctgtcatggccttaatgatct | | TEATVVTLGEDNLIIREE | |
| | atatgctgatacccagcaacgagccccaccagtccatggtcct | | QVPMELVQRGDIVKVV | |
| | ggaccacaacatcattccaggactgtccattctaaatctcatctt | | PGGKFPVDGKVLEGNT | |
| | ctttatcttgtgtacctttgtccagctcctcggtgggtggtacttc | | MADESLITGEAMPVTKK | |
| | tacgttcaggcctacaaatctctgagacacaggtcagccaaca | | PGSTVIAGSINAHGSVLI | |
| | tggacgtgctcatcgtcctggccacaagcattgcttatgtttatt | | KATHVGNDTTLAQIVKL | |
| | ctctggtcatcctggtggttgctgtggctgagaaggcggaga | | VEEAQMSKAPIQQLAD | |
| | ggagccctgtgacattcttcgacacgccccccatgctctttgtg | | RFSGYFVPFIIIMSTLTLVV | |
| | ttcattgccctgggccggtggctggaacacttggcaaagagc | | WIVIGFIDFGVVQRYFPN | |
| | aaaacctcagaagccctggctaaactcatgtctctccaagcca | | PNKHISQTEVIIRFAFQTS | |
| | cagaagccaccgttgtgacccttggtgaggacaatttaatcat | | ITVLCIACPCSLGLATPTA | |
| | cagggaggagcaagtccccatggagctggtgcagcgggg | | VMVGTGVAAQNGILIKG | |
| | cgatatcgtcaaggtggtccctgggggaaagtttccagtgga | | GKPLEMAHKIKTVMFDK | |
| | tgggaaagtcctggaaggcaataccatggctgatgagtccct | | TGTITHGVPRVMRVLLL | |
| | catcacaggagaagccatgccagtcactaagaaacccggaa | | GDVATLPLRKVLAVVGT | |
| | gcactgtaattgcggggtctataaatgcacatggctctgtgctc | | AEASSEHPLGVAVTKYC | |
| | attaaagctacccacgtgggcaatgacaccactttggctcaga | | KEELGTETLGYCTDFQA | |
| | ttgtgaaactggtggaagaggctcagatgtcaaaggcaccc | | VPGCGIGCKVSNVEGIL | |
| | attcagcagctggctgaccggtttagtggatattttgtcccattt | | AHSERPLSAPASHLNEA | |
| | atcatcatcatgtcaactttgacgttggtggtatggattgtaatc | | GSLPAEKDAVPQTFSVLI | |
| | ggttttatcgattttggtgttgttcagagatactttcctaacccca | | GNREWLRRNGLTISSDV | |
| | acaagcacatctcccagacagaggtgatcatccggtttgctttc | | SDAMTDHEMKGQTAIL | |
| | cagacgtccatcacggtgctgtgcattgcctgcccctgctccct | | VAIDGVLCGMIAIADAV | |
| | ggggctggccacgcccacggctgtcatggtgggcaccggg | | KQEAALAVHTLQSMGV | |
| | gtggccgcgcagaacggcatcctcatcaagggaggcaagc | | DVVLITGDNRKTARAIAT | |
| | ccctggagatggcgcacaagataaagactgtgatgtttgaca | | QVGINKVFAEVLPSHKV | |
| | agactggcaccattacccatggcgtccccagggtcatgcggg | | AKVQELQNKGKKVAMV | |
| | tgctcctgctgggggatgtggccacactgcccctcaggaagg | | GDGVNDSPALAQADM | |
| | ttctggctgtggtggggactgcggaggccagcagtgaacac | | GVAIGTGTDVAIEAADV | |
| | cccttgggcgtggcagtcaccaaatactgtaaagaggaactt | | VLIRNDLLDVVASIHLSK | |
| | ggaacagagaccttgggatactgcacggacttccaggcagt | | RTVRRIRINLVLALIYNLV | |
| | gccaggctgtggaattgggtgcaaagtcagcaacgtggaa | | GIPIAAGVFMPIGIVLQP | |
| | ggcatcctggcccacagtgagcgccctttgagtgcaccggcc | | WMGSAAMAASSVSVV | |
| | agtcacctgaatgaggctggcagccttcccgcagaaaaagat | | LSSLQLKCYKKPDLERYE | |
| | gcagtcccccagaccttctctgtgctgattggaaaccgtgagt | | AQAHGHMKPLTASQVS | |
| | ggctgaggcgcaacggtttaaccatttctagcgatgtcagtga | | VHIGMDDRWRDSPRAT | |
| | cgctatgacagaccacgagatgaaaggacagacagccatcc | | PWDQVSYVSQVSLSSLT | |
| | tggtggctattgacggtgtgctctgtgggatgatcgcaatcgc | | SDKPSRHSAAADDDGD | |
| | agacgctgtcaagcaggaggctgccctggctgtgcacacgc | | KWSLLLNGRDEEQYI* | |
| | tgcagagcatgggtgtggacgtggttctgatcacgggggac | | | |
| | aaccggaagacagccagagctattgccacccaggttggcat | | | |
| | caacaaagtctttgcagaggtgctgccttcgcacaaggtggc | | | |
| | caaggtccaggagctccagaataaagggaagaaagtcgcc | | | |
| | atggtgggggatggggtcaatgactccccggccttggccca | | | |
| | ggcagacatgggtgtggccattggcaccggcacggatgtg | | | |
| | gccatcgaggcagccgacgtcgtccttatcagaaatgatttgc | | | |
| | tggatgtggtggctagcattcacctttccaagaggactgtccg | | | |
| | aaggatacgcatcaacctggtcctggcactgatttataacctg | | | |
| | gttgggatacccattgcagcaggtgtcttcatgcccatcggca | | | |
| | ttgtgctgcagccctggatgggctcagcggccatggcagcct | | | |
| | cctctgtgtctgtggtgctctcatccctgcagctcaagtgctata | | | |
| | agaagcctgacctggagaggtatgaggcacaggcgcatgg | | | |
| | ccacatgaagcccctgacggcatcccaggtcagtgtgcacat | | | |
| | aggcatggatgacaggtggcgggactcccccagggccaca | | | |
| | ccatgggaccaggtcagctatgtcagccaggtgtcgctgtcct | | | |
| | ccctgacgtccgacaagccatctcggcacaggctgcagca | | | |
| | gacgatgatggggacaagtggtctctgctcctgaatggcag | | | |
| | ggatgaggagcagtacatctga | | | |
| Truncated | atgcctgagcaggagagacagatcacagccagagaaggg | 17 | MPEQERQITAREGASRKI | 18 |
| ATP7B | gccagtcggaaaatcttatctaagctttctttgcctacccgtgcc | | LSKLSLPTRAWEPAMKK | |
| (CRE1 and | tgggaaccagcaatgaagaagagttttgcttttgacaatgttg | | SFAFDNVGYEGGLDGLG | |
| CRE5 are | gctatgaaggtggtctggatggcctgggcccttcttctcaggt | | PSSQVATSTVRILGMTC | |
| present) | ggccaccagcacagtcaggatcttgggcatgacttgccagtc | | QSCVKSIEDRISNLKGIIS | |
| | atgtgtgaagtccattgaggacaggatttccaatttgaaaggc | | MKVSLEQGSATVKYVPS | |
| | atcatcagcatgaaggtttccctggaacaaggcagtgccact | | VVCLQQVCHQIGDMGF | |
| | gtgaaatatgtgccatcggttgtgtgcctgcaacaggtttgcc | | EASIVSESCSTNPLGNHS | |
| | atcaaattggggacatgggcttcgaggccagcattgtttctga | | AGNSMVQTTDGTPTSV | |
| | aagctgttctactaaccctcttggaaaccacagtgctgggaatt | | QEVAPHTGRLPANHAP | |
| | ccatggtgcaaactacagatggtacacctacatctgtgcagg | | DILAKSPQSTRAVAPQK | |
| | aagtggctccccacactgggaggctccctgcaaaccatgccc | | CFLQIKGMTCASCVSNIE | |
| | cggacatcttggcaaagtccccacaatcaaccagagcagtgg | | RNLQKEAGVLSVLVALM | |
| | caccgcagaagtgcttcttacagatcaaaggcatgacctgtgc | | AGKAEIKYDPEVIQPLEIA | |
| | atcctgtgtgtctaacatagaaaggaatctgcagaaagaagc | | QFIQDLGFEAAVAQRNP | |
| | tggtgttctctccgtgttggttgccttgatggcaggaaaggca | | NAHHLDHKMEIKQWKK | |
| | gagatcaagtatgacccagaggtcatccagcccctcgagata | | SFLCSLVFGIPVMALMIY | |
| | gctcagttcatccaggacctgggttttgaggcagcagtcgccc | | MLIPSNEPHQSMVLDH | |
| | agagaaaccccaacgctcatcacttggaccacaagatggaa | | NIIPGLSILNLIFFILCTFV | |
| | ataaagcagtggaagaagtctttcctgtgcagcctggtgtttg | | QLLGGWYFYVQAYKSLR | |
| | gcatccctgtcatggccttaatgatctatatgctgatacccagc | | HRSANMDVLIVLATSIAY | |
| | aacgagccccaccagtccatggtcctggaccacaacatcattcca | | VYSLVILVVAVAEKAERS | |
| | ggactgtccattctaaatctcatcttctttatcttgtgtacctttg | | PVTFFDTPPMLFVFIALG | |
| | tccagctcctcggtgggtggtacttctacgttcaggcctacaaa | | RWLEHLAKSKTSEALAKL | |
| | tctctgagacacaggtcagccaacatggacgtgctcatcgtcc | | MSLQATEATVVTLGEDN | |
| | tggccacaagcattgcttatgtttattctctggtcatcctggtggt | | LIIREEQVPMELVQRGDI | |
| | tgctgtggctgagaaggcggagaggagccctgtgacattctt | | VKVVPGGKFPVDGKVLE | |
| | cgacacgccccccatgctctttgtgttcattgccctgggccggt | | GNTMADESLITGEAMPV | |
| | ggctggaacacttggcaaagagcaaaacctcagaagccctg | | TKKPGSTVIAGSINAHGS | |
| | gctaaactcatgtctctccaagccacagaagccaccgttgtga | | VLIKATHVGNDTTLAQIV | |
| | cccttggtgaggacaatttaatcatcagggaggagcaagtcc | | KLVEEAQMSKAPIQQLA | |
| | ccatggagctggtgcagcggggcgatatcgtcaaggtggtc | | DRFSGYFVPFIIIMSTLTL | |
| | cctgggggaaagtttccagtggatgggaaagtcctggaagg | | VVWIVIGFIDFGVVQRYF | |
| | caataccatggctgatgagtccctcatcacaggagaagccat | | PNPNKHISQTEVIIRFAF | |
| | gccagtcactaagaaacccggaagcactgtaattgcggggt | | QTSITVLCIACPCSLGLAT | |
| | ctataaatgcacatggctctgtgctcattaaagctacccacgtg | | PTAVMVGTGVAAQNGI | |
| | ggcaatgacaccactttggctcagattgtgaaactggtggaa | | LIKGGKPLEMAHKIKTV | |
| | gaggctcagatgtcaaaggcacccattcagcagctggctgac | | MFDKTGTITHGVPRVM | |
| | cggtttagtggatattttgtcccatttatcatcatcatgtcaacttt | | RVLLLGDVATLPLRKVLA | |
| | gacgttggtggtatggattgtaatcggttttatcgattttggtgt | | VVGTAEASSEHPLGVAV | |
| | tgttcagagatactttcctaaccccaacaagcacatctcccaga | | TKYCKEELGTETLGYCTD | |
| | cagaggtgatcatccggtttgctttccagacgtccatcacggt | | FQAVPGCGIGCKVSNVE | |
| | gctgtgcattgcctgcccctgctccctggggctggccacgccc | | GILAHSERPLSAPASHLN | |
| | acggctgtcatggtgggcaccggggtggccgcgcagaacg | | EAGSLPAEKDAVPQTFS | |
| | gcatcctcatcaagggaggcaagcccctggagatggcgcac | | VLIGNREWLRRNGLTISS | |
| | aagataaagactgtgatgtttgacaagactggcaccattaccc | | DVSDAMTDHEMKGQT | |
| | atggcgtccccagggtcatgcgggtgctcctgctgggggat | | AILVAIDGVLCGMIAIAD | |
| | gtggccacactgcccctcaggaaggttctggctgtggtggg | | AVKQEAALAVHTLQSM | |
| | gactgcggaggccagcagtgaacaccccttgggcgtggca | | GVDVVLITGDNRKTARA | |
| | gtcaccaaatactgtaaagaggaacttggaacagagaccttg | | IATQVGINKVFAEVLPSH | |
| | ggatactgcacggacttccaggcagtgccaggctgtggaatt | | KVAKVQELQNKGKKVA | |
| | gggtgcaaagtcagcaacgtggaaggcatcctggcccaca | | MVGDGVNDSPALAQA | |
| | gtgagcgccctttgagtgcaccggccagtcacctgaatgagg | | DMGVAIGTGTDVAIEAA | |
| | ctggcagccttcccgcagaaaaagatgcagtcccccagacct | | DVVLIRNDLLDVVASIHL | |
| | tctctgtgctgattggaaaccgtgagtggctgaggcgcaacg | | SKRTVRRIRINLVLALIYN | |
| | gtttaaccatttctagcgatgtcagtgacgctatgacagaccac | | LVGIPIAAGVFMPIGIVL | |
| | gagatgaaaggacagacagccatcctggtggctattgacgg | | QPWMGSAAMAASSVS | |
| | tgtgctctgtgggatgatcgcaatcgcagacgctgtcaagca | | VVLSSLQLKCYKKPDLER | |
| | ggaggctgccctggctgtgcacacgctgcagagcatgggtg | | YEAQAHGHMKPLTASQ | |
| | tggacgtggttctgatcacgggggacaaccggaagacagcc | | VSVHIGMDDRWRDSPR | |
| | agagctattgccacccaggttggcatcaacaaagtctttgcag | | ATPWDQVSYVSQVSLS | |
| | aggtgctgccttcgcacaaggtggccaaggtccaggagctc | | SLTSDKPSRHSAAADDD | |
| | cagaataaagggaagaaagtcgccatggtgggggatggg | | GDKWSLLLNGRDEEQYI | |
| | gtcaatgactccccggccttggcccaggcagacatgggtgtg | | * | |
| | gccattggcaccggcacggatgtggccatcgaggcagccg | | | |
| | acgtcgtccttatcagaaatgatttgctggatgtggtggctagc | | | |
| | attcacctttccaagaggactgtccgaaggatacgcatcaacc | | | |
| | tggtcctggcactgatttataacctggttgggatacccattgca | | | |
| | gcaggtgtcttcatgcccatcggcattgtgctgcagccctgga | | | |
| | tgggctcagcggccatggcagcctcctctgtgtctgtggtgct | | | |
| | ctcatccctgcagctcaagtgctataagaagcctgacctggag | | | |
| | aggtatgaggcacaggcgcatggccacatgaagcccctgac | | | |
| | ggcatcccaggtcagtgtgcacataggcatggatgacaggt | | | |
| | ggcgggactcccccagggccacaccatgggaccaggtcag | | | |
| | ctatgtcagccaggtgtcgctgtcctccctgacgtccgacaag | | | |
| | ccatctcggcacagcgctgcagcagacgatgatggggacaa | | | |
| | gtggtctctgctcctgaatggcagggatgaggagcagtacat | | | |
| | ctga | | | |
| Truncated | atgcctgagcaggagagacagatcacagccagagaaggg | 19 | MPEQERQITAREGASRKI | 20 |
| ATP7B | gccagtcggaaaatcttatctaagctttctttgcctacccgtgcc | | LSKLSLPTRAWEPAMKK | |
| (CRE1 and | tgggaaccagcaatgaagaagagttttgcttttgacaatgttg | | SFAFDNVGYEGGLDGLG | |
| CRE6 are | gctatgaaggtggtctggatggcctgggcccttcttctcaggt | | PSSQVATSTVRILGMTC | |
| present) | ggccaccagcacagtcaggatcttgggcatgacttgccagtc | | QSCVKSIEDRISNLKGIIS | |
| | atgtgtgaagtccattgaggacaggatttccaatttgaaaggc | | MKVSLEQGSATVKYVPS | |
| | atcatcagcatgaaggtttccctggaacaaggcagtgccact | | VVCLQQVCHQIGDMGF | |
| | gtgaaatatgtgccatcggttgtgtgcctgcaacaggtttgcc | | EASIMEDYAGSDGNIELT | |
| | atcaaattggggacatgggcttcgaggccagcattatggag | | ITGMTCASCVHNIESKLT | |
| | gactacgcaggctccgatggcaacattgagctgacaatcaca | | RTNGITYASVALATSKAL | |
| | gggatgacctgcgcgtcctgtgtccacaacatagagtccaaa | | VKFDPEIIGPRDIIKIIEEIG | |
| | ctcacgaggacaaatggcatcacttatgcctccgttgcccttgc | | FHASLAQRNPNAHHLD | |
| | caccagcaaagcccttgttaagtttgacccggaaattatcggt | | HKMEIKQWKKSFLCSLV | |
| | ccacgggatattatcaaaattattgaggaaattggctttcatgc | | FGIPVMALMIYMLIPSNE | |
| | ttccctggcccagagaaaccccaacgctcatcacttggaccac | | PHQSMVLDHNIIPGLSIL | |
| | aagatggaaataaagcagtggaagaagtctttcctgtgcagc | | NLIFFILCTFVQLLGGWY | |
| | ctggtgtttggcatccctgtcatggccttaatgatctatatgctg | | FYVQAYKSLRHRSANM | |
| | atacccagcaacgagccccaccagtccatggtcctggaccac | | DVLIVLATSIAYVYSLVIL | |
| | aacatcattccaggactgtccattctaaatctcatcttctttatctt | | VVAVAEKAERSPVTFFD | |
| | gtgtacctttgtccagctcctcggtgggtggtacttctacgttca | | TPPMLFVFIALGRWLEHL | |
| | ggcctacaaatctctgagacacaggtcagccaacatggacgt | | AKSKTSEALAKLMSLQA | |
| | gctcatcgtcctggccacaagcattgcttatgtttattctctggtc | | TEATVVTLGEDNLIIREE | |
| | atcctggtggttgctgtggctgagaaggcggagaggagccc | | QVPMELVQRGDIVKVV | |
| | tgtgacattcttcgacacgccccccatgctctttgtgttcattgcc | | PGGKFPVDGKVLEGNT | |
| | ctgggccggtggctggaacacttggcaaagagcaaaacctc | | MADESLITGEAMPVTKK | |
| | agaagccctggctaaactcatgtctctccaagccacagaagc | | PGSTVIAGSINAHGSVLI | |
| | caccgttgtgacccttggtgaggacaatttaatcatcagggag | | KATHVGNDTTLAQIVKL | |
| | gagcaagtccccatggagctggtgcagcggggcgatatcgt | | VEEAQMSKAPIQQLAD | |
| | caaggtggtccctgggggaaagtttccagtggatgggaaag | | RFSGYFVPFIIIMSTLTLVV | |
| | tcctggaaggcaataccatggctgatgagtccctcatcacag | | WIVIGFIDFGVVQRYFPN | |
| | gagaagccatgccagtcactaagaaacccggaagcactgta | | PNKHISQTEVIIRFAFQTS | |
| | attgcggggtctataaatgcacatggctctgtgctcattaaagc | | ITVLCIACPCSLGLATPTA | |
| | tacccacgtgggcaatgacaccactttggctcagattgtgaaa | | VMVGTGVAAQNGILIKG | |
| | ctggtggaagaggctcagatgtcaaaggcacccattcagca | | GKPLEMAHKIKTVMFDK | |
| | gctggctgaccggtttagtggatattttgtcccatttatcatcatc | | TGTITHGVPRVMRVLLL | |
| | atgtcaactttgacgttggtggtatggattgtaatcggttttatc | | GDVATLPLRKVLAVVGT | |
| | gattttggtgttgttcagagatactttcctaaccccaacaagcac | | AEASSEHPLGVAVTKYC | |
| | atctcccagacagaggtgatcatccggtttgctttccagacgtc | | KEELGTETLGYCTDFQA | |
| | catcacggtgctgtgcattgcctgcccctgctccctggggctg | | VPGCGIGCKVSNVEGIL | |
| | gccacgcccacggctgtcatggtgggcaccggggtggccg | | AHSERPLSAPASHLNEA | |
| | cgcagaacggcatcctcatcaagggaggcaagcccctgga | | GSLPAEKDAVPQTFSVLI | |
| | gatggcgcacaagataaagactgtgatgtttgacaagactgg | | GNREWLRRNGLTISSDV | |
| | caccattacccatggcgtccccagggtcatgcgggtgctcctg | | SDAMTDHEMKGQTAIL | |
| | ctgggggatgtggccacactgcccctcaggaaggttctggct | | VAIDGVLCGMIAIADAV | |
| | gtggtggggactgcggaggccagcagtgaacaccccttgg | | KQEAALAVHTLQSMGV | |
| | gcgtggcagtcaccaaatactgtaaagaggaacttggaaca | | DVVLITGDNRKTARAIAT | |
| | gagaccttgggatactgcacggacttccaggcagtgccagg | | QVGINKVFAEVLPSHKV | |
| | ctgtggaattgggtgcaaagtcagcaacgtggaaggcatcct | | AKVQELQNKGKKVAMV | |
| | ggcccacagtgagcgccctttgagtgcaccggccagtcacct | | GDGVNDSPALAQADM | |
| | gaatgaggctggcagccttcccgcagaaaaagatgcagtcc | | GVAIGTGTDVAIEAADV | |
| | cccagaccttctctgtgctgattggaaaccgtgagtggctgag | | VLIRNDLLDVVASIHLSK | |
| | gcgcaacggtttaaccatttctagcgatgtcagtgacgctatg | | RTVRRIRINLVLALIYNLV | |
| | acagaccacgagatgaaaggacagacagccatcctggtgg | | GIPIAAGVFMPIGIVLQP | |
| | ctattgacggtgtgctctgtgggatgatcgcaatcgcagacgc | | WMGSAAMAASSVSVV | |
| | tgtcaagcaggaggctgccctggctgtgcacacgctgcaga | | LSSLQLKCYKKPDLERYE | |
| | gcatgggtgtggacgtggttctgatcacgggggacaaccgg | | AQAHGHMKPLTASQVS | |
| | aagacagccagagctattgccacccaggttggcatcaacaaa | | VHIGMDDRWRDSPRAT | |
| | gtctttgcagaggtgctgccttcgcacaaggtggccaaggtc | | PWDQVSYVSQVSLSSLT | |
| | caggagctccagaataaagggaagaaagtcgccatggtgg | | SDKPSRHSAAADDDGD | |
| | gggatggggtcaatgactccccggccttggcccaggcagac | | KWSLLLNGRDEEQYI* | |
| | atgggtgtggccattggcaccggcacggatgtggccatcga | | | |
| | ggcagccgacgtcgtccttatcagaaatgatttgctggatgtg | | | |
| | gtggctagcattcacctttccaagaggactgtccgaaggatac | | | |
| | gcatcaacctggtcctggcactgatttataacctggttgggata | | | |
| | cccattgcagcaggtgtcttcatgcccatcggcattgtgctgca | | | |
| | gccctggatgggctcagcggccatggcagcctcctctgtgtct | | | |
| | gtggtgctctcatccctgcagctcaagtgctataagaagcctg | | | |
| | acctggagaggtatgaggcacaggcgcatggccacatgaa | | | |
| | gcccctgacggcatcccaggtcagtgtgcacataggcatgg | | | |
| | atgacaggtggcgggactcccccagggccacaccatggga | | | |
| | ccaggtcagctatgtcagccaggtgtcgctgtcctccctgacg | | | |
| | tccgacaagccatctcggcacagcgctgcagcagacgatgat | | | |
| | ggggacaagtggtctctgctcctgaatggcagggatgagga | | | |
| | gcagtacatctga | | | |
| Truncated | atgcctgagcaggagagacagatcacagccagagaaggg | 21 | MPEQERQITAREGASRKI | 22 |
| ATP7B | gccagtcggaaaatcttatctaagctttctttgcctacccgtgcc | | LSKLSLPTRAWEPAMKK | |
| (CRE1, | tgggaaccagcaatgaagaagagttttgcttttgacaatgttg | | SFAFDNVGYEGGLDGLG | |
| CRE5, and | gctatgaaggtggtctggatggcctgggcccttcttctcaggt | | PSSQVATSTVRILGMTC | |
| CRE6 are | ggccaccagcacagtcaggatcttgggcatgacttgccagtc | | QSCVKSIEDRISNLKGIIS | |
| present) | atgtgtgaagtccattgaggacaggatttccaatttgaaaggc | | MKVSLEQGSATVKYVPS | |
| | atcatcagcatgaaggtttccctggaacaaggcagtgccact | | VVCLQQVCHQIGDMGF | |
| | gtgaaatatgtgccatcggttgtgtgcctgcaacaggtttgcc | | EASIVSESCSTNPLGNHS | |
| | atcaaattggggacatgggcttcgaggccagcattgtttctga | | AGNSMVQTTDGTPTSV | |
| | aagctgttctactaaccctcttggaaaccacagtgctgggaatt | | QEVAPHTGRLPANHAP | |
| | ccatggtgcaaactacagatggtacacctacatctgtgcagg | | DILAKSPQSTRAVAPQK | |
| | aagtggctccccacactgggaggctccctgcaaaccatgccc | | CFLQIKGMTCASCVSNIE | |
| | cggacatcttggcaaagtccccacaatcaaccagagcagtgg | | RNLQKEAGVLSVLVALM | |
| | caccgcagaagtgcttcttacagatcaaaggcatgacctgtgc | | AGKAEIKYDPEVIQPLEIA | |
| | atcctgtgtgtctaacatagaaaggaatctgcagaaagaagc | | QFIQDLGFEAAVMEDYA | |
| | tggtgttctctccgtgttggttgccttgatggcaggaaaggca | | GSDGNIELTITGMTCAS | |
| | gagatcaagtatgacccagaggtcatccagcccctcgagata | | CVHNIESKLTRTNGITYA | |
| | gctcagttcatccaggacctgggttttgaggcagcagtcatgg | | SVALATSKALVKFDPEIIG | |
| | aggactacgcaggctccgatggcaacattgagctgacaatca | | PRDIIKIIEEIGFHASLAQR | |
| | cagggatgacctgcgcgtcctgtgtccacaacatagagtcca | | NPNAHHLDHKMEIKQ | |
| | aactcacgaggacaaatggcatcacttatgcctccgttgccctt | | WKKSFLCSLVFGIPVMAL | |
| | gccaccagcaaagcccttgttaagtttgacccggaaattatcg | | MIYMLIPSNEPHQSMVL | |
| | gtccacgggatattatcaaaattattgaggaaattggctttcat | | DHNIIPGLSILNLIFFILCT | |
| | gcttccctggcccagagaaaccccaacgctcatcacttggacc | | FVQLLGGWYFYVQAYK | |
| | acaagatggaaataaagcagtggaagaagtctttcctgtgca | | SLRHRSANMDVLIVLAT | |
| | gcctggtgtttggcatccctgtcatggccttaatgatctatatgc | | SIAYVYSLVILVVAVAEKA | |
| | tgatacccagcaacgagccccaccagtccatggtcctggacc | | ERSPVTFFDTPPMLFVFI | |
| | acaacatcattccaggactgtccattctaaatctcatcttctttatc | | ALGRWLEHLAKSKTSEA | |
| | ttgtgtacctttgtccagctcctcggtgggtggtacttctacgttc | | LAKLMSLQATEATVVTL | |
| | aggcctacaaatctctgagacacaggtcagccaacatggac | | GEDNLIIREEQVPMELV | |
| | gtgctcatcgtcctggccacaagcattgcttatgtttattctctg | | QRGDIVKVVPGGKFPVD | |
| | gtcatcctggtggttgctgtggctgagaaggcggagaggag | | GKVLEGNTMADESLITG | |
| | ccctgtgacattcttcgacacgccccccatgctctttgtgttcatt | | EAMPVTKKPGSTVIAGSI | |
| | gccctgggccggtggctggaacacttggcaaagagcaaaa | | NAHGSVLIKATHVGNDT | |
| | cctcagaagccctggctaaactcatgtctctccaagccacaga | | TLAQIVKLVEEAQMSKA | |
| | agccaccgttgtgacccttggtgaggacaatttaatcatcagg | | PIQQLADRESGYFVPFIII | |
| | gaggagcaagtccccatggagctggtgcagcggggcgata | | MSTLTLVVWIVIGFIDFG | |
| | tcgtcaaggtggtccctgggggaaagtttccagtggatggg | | VVQRYFPNPNKHISQTE | |
| | aaagtcctggaaggcaataccatggctgatgagtccctcatc | | VIIRFAFQTSITVLCIACPC | |
| | acaggagaagccatgccagtcactaagaaacccggaagca | | SLGLATPTAVMVGTGVA | |
| | ctgtaattgcggggtctataaatgcacatggctctgtgctcatt | | AQNGILIKGGKPLEMAH | |
| | aaagctacccacgtgggcaatgacaccactttggctcagattg | | KIKTVMFDKTGTITHGVP | |
| | tgaaactggtggaagaggctcagatgtcaaaggcacccattc | | RVMRVLLLGDVATLPLR | |
| | agcagctggctgaccggtttagtggatattttgtcccatttatca | | KVLAVVGTAEASSEHPL | |
| | tcatcatgtcaactttgacgttggtggtatggattgtaatcggtt | | GVAVTKYCKEELGTETLG | |
| | ttatcgattttggtgttgttcagagatactttcctaaccccaacaa | | YCTDFQAVPGCGIGCKV | |
| | gcacatctcccagacagaggtgatcatccggtttgctttccag | | SNVEGILAHSERPLSAPA | |
| | acgtccatcacggtgctgtgcattgcctgcccctgctccctgg | | SHLNEAGSLPAEKDAVP | |
| | ggctggccacgcccacggctgtcatggtgggcaccggggt | | QTFSVLIGNREWLRRNG | |
| | ggccgcgcagaacggcatcctcatcaagggaggcaagccc | | LTISSDVSDAMTDHEMK | |
| | ctggagatggcgcacaagataaagactgtgatgtttgacaag | | GQTAILVAIDGVLCGMI | |
| | actggcaccattacccatggcgtccccagggtcatgcgggtg | | AIADAVKQEAALAVHTL | |
| | ctcctgctgggggatgtggccacactgcccctcaggaaggtt | | QSMGVDVVLITGDNRK | |
| | ctggctgtggtggggactgcggaggccagcagtgaacacc | | TARAIATQVGINKVFAEV | |
| | ccttgggcgtggcagtcaccaaatactgtaaagaggaacttg | | LPSHKVAKVQELQNKGK | |
| | gaacagagaccttgggatactgcacggacttccaggcagtg | | KVAMVGDGVNDSPALA | |
| | ccaggctgtggaattgggtgcaaagtcagcaacgtggaag | | QADMGVAIGTGTDVAIE | |
| | gcatcctggcccacagtgagcgccctttgagtgcaccggcca | | AADVVLIRNDLLDVVASI | |
| | gtcacctgaatgaggctggcagccttcccgcagaaaaagat | | HLSKRTVRRIRINLVLALI | |
| | gcagtcccccagaccttctctgtgctgattggaaaccgtgagt | | YNLVGIPIAAGVFMPIGI | |
| | ggctgaggcgcaacggtttaaccatttctagcgatgtcagtga | | VLQPWMGSAAMAASS | |
| | cgctatgacagaccacgagatgaaaggacagacagccatcc | | VSVVLSSLQLKCYKKPDL | |
| | tggtggctattgacggtgtgctctgtgggatgatcgcaatcgc | | ERYEAQAHGHMKPLTA | |
| | agacgctgtcaagcaggaggctgccctggctgtgcacacgc | | SQVSVHIGMDDRWRDS | |
| | tgcagagcatgggtgtggacgtggttctgatcacgggggac | | PRATPWDQVSYVSQVS | |
| | aaccggaagacagccagagctattgccacccaggttggcat | | LSSLTSDKPSRHSAAAD | |
| | caacaaagtctttgcagaggtgctgccttcgcacaaggtggc | | DDGDKWSLLLNGRDEE | |
| | caaggtccaggagctccagaataaagggaagaaagtcgcc | | QYl* | |
| | atggtgggggatggggtcaatgactccccggccttggccca | | | |
| | ggcagacatgggtgtggccattggcaccggcacggatgtg | | | |
| | gccatcgaggcagccgacgtcgtccttatcagaaatgatttgc | | | |
| | tggatgtggtggctagcattcacctttccaagaggactgtccg | | | |
| | aaggatacgcatcaacctggtcctggcactgatttataacctg | | | |
| | gttgggatacccattgcagcaggtgtcttcatgcccatcggca | | | |
| | ttgtgctgcagccctggatgggctcagcggccatggcagcct | | | |
| | cctctgtgtctgtggtgctctcatccctgcagctcaagtgctata | | | |
| | agaagcctgacctggagaggtatgaggcacaggcgcatgg | | | |
| | ccacatgaagcccctgacggcatcccaggtcagtgtgcacat | | | |
| | aggcatggatgacaggtggcgggactcccccagggccaca | | | |
| | ccatgggaccaggtcagctatgtcagccaggtgtcgctgtcct | | | |
| | ccctgacgtccgacaagccatctcggcacagcgctgcagca | | | |
| | gacgatgatggggacaagtggtctctgctcctgaatggcag | | | |
| | ggatgaggagcagtacatctga | | | |
| Truncated | atgcctgagcaggagagacagatcacagccagagaaggg | 23 | MPEQERQITAREGASRKI | 24 |
| ATP7B | gccagtcggaaaatcttatctaagctttctttgcctacccgtgcc | | LSKLSLPTRAWEPAMKK | |
| (CRE2 and | tgggaaccagcaatgaagaagagttttgcttttgacaatgttg | | SFAFDNVGYEGGLDGLG | |
| CRE5 are | gctatgaaggtggtctggatggcctgggcccttcttctcaggt | | PSSQVATSTVRVVKLRV | |
| present) | ggccaccagcacagtcagggtggtcaagctccgggtggag | | EGMTCQSCVSSIEGKVR | |
| | ggcatgacctgccagtcctgtgtcagctccattgaaggcaag | | KLQGVVRVKVSLSNQEA | |
| | gtccggaaactgcaaggagtagtgagagtcaaagtctcactc | | VITYQPYLIQPEDLRDHV | |
| | agcaaccaagaggccgtcatcacttatcagccttatctcattca | | NDMGFEAAIVSESCSTN | |
| | gcccgaagacctcagggaccatgtaaatgacatgggatttga | | PLGNHSAGNSMVQTTD | |
| | agctgccatcgtttctgaaagctgttctactaaccctcttggaaa | | GTPTSVQEVAPHTGRLP | |
| | ccacagtgctgggaattccatggtgcaaactacagatggtac | | ANHAPDILAKSPQSTRA | |
| | acctacatctgtgcaggaagtggctccccacactgggaggct | | VAPQKCFLQIKGMTCAS | |
| | ccctgcaaaccatgccccggacatcttggcaaagtccccaca | | CVSNIERNLQKEAGVLS | |
| | atcaaccagagcagtggcaccgcagaagtgcttcttacagat | | VLVALMAGKAEIKYDPE | |
| | caaaggcatgacctgtgcatcctgtgtgtctaacatagaaag | | VIQPLEIAQFIQDLGFEA | |
| | gaatctgcagaaagaagctggtgttctctccgtgttggttgcct | | AVAQRNPNAHHLDHK | |
| | tgatggcaggaaaggcagagatcaagtatgacccagaggt | | MEIKQWKKSFLCSLVFGI | |
| | catccagcccctcgagatagctcagttcatccaggacctgggt | | PVMALMIYMLIPSNEPH | |
| | tttgaggcagcagtcgcccagagaaaccccaacgctcatcac | | QSMVLDHNIIPGLSILNLI | |
| | ttggaccacaagatggaaataaagcagtggaagaagtctttc | | FFILCTFVQLLGGWYFYV | |
| | ctgtgcagcctggtgtttggcatccctgtcatggccttaatgat | | QAYKSLRHRSANMDVLI | |
| | ctatatgctgatacccagcaacgagccccaccagtccatggtc | | VLATSIAYVYSLVILVVAV | |
| | ctggaccacaacatcattccaggactgtccattctaaatctcatc | | AEKAERSPVTFFDTPPML | |
| | ttctttatcttgtgtacctttgtccagctcctcggtgggtggtactt | | FVFIALGRWLEHLAKSKT | |
| | ctacgttcaggcctacaaatctctgagacacaggtcagccaac | | SEALAKLMSLQATEATV | |
| | atggacgtgctcatcgtcctggccacaagcattgcttatgtttat | | VTLGEDNLIIREEQVPME | |
| | tctctggtcatcctggtggttgctgtggctgagaaggcggag | | LVQRGDIVKVVPGGKFP | |
| | aggagccctgtgacattcttcgacacgccccccatgctctttgt | | VDGKVLEGNTMADESLI | |
| | gttcattgccctgggccggtggctggaacacttggcaaagag | | TGEAMPVTKKPGSTVIA | |
| | caaaacctcagaagccctggctaaactcatgtctctccaagcc | | GSINAHGSVLIKATHVG | |
| | acagaagccaccgttgtgacccttggtgaggacaatttaatca | | NDTTLAQIVKLVEEAQM | |
| | tcagggaggagcaagtccccatggagctggtgcagcgggg | | SKAPIQQLADRESGYFV | |
| | cgatatcgtcaaggtggtccctgggggaaagtttccagtgga | | PFIIIMSTLTLVVWIVIGFI | |
| | tgggaaagtcctggaaggcaataccatggctgatgagtccct | | DFGVVQRYFPNPNKHIS | |
| | catcacaggagaagccatgccagtcactaagaaacccggaa | | QTEVIIRFAFQTSITVLCIA | |
| | gcactgtaattgcggggtctataaatgcacatggctctgtgctc | | CPCSLGLATPTAVMVGT | |
| | attaaagctacccacgtgggcaatgacaccactttggctcaga | | GVAAQNGILIKGGKPLE | |
| | ttgtgaaactggtggaagaggctcagatgtcaaaggcaccc | | MAHKIKTVMFDKTGTIT | |
| | attcagcagctggctgaccggtttagtggatattttgtcccattt | | HGVPRVMRVLLLGDVA | |
| | atcatcatcatgtcaactttgacgttggtggtatggattgtaatc | | TLPLRKVLAVVGTAEASS | |
| | ggttttatcgattttggtgttgttcagagatactttcctaacccca | | EHPLGVAVTKYCKEELGT | |
| | acaagcacatctcccagacagaggtgatcatccggtttgctttc | | ETLGYCTDFQAVPGCGI | |
| | cagacgtccatcacggtgctgtgcattgcctgcccctgctccct | | GCKVSNVEGILAHSERPL | |
| | ggggctggccacgcccacggctgtcatggtgggcaccggg | | SAPASHLNEAGSLPAEK | |
| | gtggccgcgcagaacggcatcctcatcaagggaggcaagc | | DAVPQTFSVLIGNREWL | |
| | ccctggagatggcgcacaagataaagactgtgatgtttgaca | | RRNGLTISSDVSDAMTD | |
| | agactggcaccattacccatggcgtccccagggtcatgcggg | | HEMKGQTAILVAIDGVL | |
| | tgctcctgctgggggatgtggccacactgcccctcaggaagg | | CGMIAIADAVKQEAALA | |
| | ttctggctgtggtggggactgcggaggccagcagtgaacac | | VHTLQSMGVDVVLITGD | |
| | cccttgggcgtggcagtcaccaaatactgtaaagaggaactt | | NRKTARAIATQVGINKV | |
| | ggaacagagaccttgggatactgcacggacttccaggcagt | | FAEVLPSHKVAKVQELQ | |
| | gccaggctgtggaattgggtgcaaagtcagcaacgtggaa | | NKGKKVAMVGDGVND | |
| | ggcatcctggcccacagtgagcgccctttgagtgcaccggcc | | SPALAQADMGVAIGTG | |
| | agtcacctgaatgaggctggcagccttcccgcagaaaaagat | | TDVAIEAADVVLIRNDLL | |
| | gcagtcccccagaccttctctgtgctgattggaaaccgtgagt | | DVVASIHLSKRTVRRIRIN | |
| | ggctgaggcgcaacggtttaaccatttctagcgatgtcagtga | | LVLALIYNLVGIPIAAGVF | |
| | cgctatgacagaccacgagatgaaaggacagacagccatcc | | MPIGIVLQPWMGSAAM | |
| | tggtggctattgacggtgtgctctgtgggatgatcgcaatcgc | | AASSVSVVLSSLQLKCYK | |
| | agacgctgtcaagcaggaggctgccctggctgtgcacacgc | | KPDLERYEAQAHGHMK | |
| | tgcagagcatgggtgtggacgtggttctgatcacgggggac | | PLTASQVSVHIGMDDR | |
| | aaccggaagacagccagagctattgccacccaggttggcat | | WRDSPRATPWDQVSYV | |
| | caacaaagtctttgcagaggtgctgccttcgcacaaggtggc | | SQVSLSSLTSDKPSRHSA | |
| | caaggtccaggagctccagaataaagggaagaaagtcgcc | | AADDDGDKWSLLLNGR | |
| | atggtgggggatggggtcaatgactccccggccttggccca | | DEEQYI* | |
| | ggcagacatgggtgtggccattggcaccggcacggatgtg | | | |
| | gccatcgaggcagccgacgtcgtccttatcagaaatgatttgc | | | |
| | tggatgtggtggctagcattcacctttccaagaggactgtccg | | | |
| | aaggatacgcatcaacctggtcctggcactgatttataacctg | | | |
| | gttgggatacccattgcagcaggtgtcttcatgcccatcggca | | | |
| | ttgtgctgcagccctggatgggctcagcggccatggcagcct | | | |
| | cctctgtgtctgtggtgctctcatccctgcagctcaagtgctata | | | |
| | agaagcctgacctggagaggtatgaggcacaggcgcatgg | | | |
| | ccacatgaagcccctgacggcatcccaggtcagtgtgcacat | | | |
| | aggcatggatgacaggtggcgggactcccccagggccaca | | | |
| | ccatgggaccaggtcagctatgtcagccaggtgtcgctgtcct | | | |
| | ccctgacgtccgacaagccatctcggcacagcgctgcagca | | | |
| | gacgatgatggggacaagtggtctctgctcctgaatggcag | | | |
| | ggatgaggagcagtacatctga | | | |
| Truncated | atgcctgagcaggagagacagatcacagccagagaaggg | 25 | MPEQERQITAREGASRKI | 26 |
| ATP7B | gccagtcggaaaatcttatctaagctttctttgcctacccgtgcc | | LSKLSLPTRAWEPAMKK | |
| (CRE2 and | tgggaaccagcaatgaagaagagttttgcttttgacaatgttg | | SFAFDNVGYEGGLDGLG | |
| CRE6 are | gctatgaaggtggtctggatggcctgggcccttcttctcaggt | | PSSQVATSTVRVVKLRV | |
| present) | ggccaccagcacagtcagggtggtcaagctccgggtggag | | EGMTCQSCVSSIEGKVR | |
| | ggcatgacctgccagtcctgtgtcagctccattgaaggcaag | | KLQGVVRVKVSLSNQEA | |
| | gtccggaaactgcaaggagtagtgagagtcaaagtctcactc | | VITYQPYLIQPEDLRDHV | |
| | agcaaccaagaggccgtcatcacttatcagccttatctcattca | | NDMGFEAAIMEDYAGS | |
| | gcccgaagacctcagggaccatgtaaatgacatgggatttga | | DGNIELTITGMTCASCV | |
| | agctgccatcatggaggactacgcaggctccgatggcaacat | | HNIESKLTRTNGITYASV | |
| | tgagctgacaatcacagggatgacctgcgcgtcctgtgtcca | | ALATSKALVKFDPEIIGPR | |
| | caacatagagtccaaactcacgaggacaaatggcatcacttat | | DIIKIIEEIGFHASLAQRNP | |
| | gcctccgttgcccttgccaccagcaaagcccttgttaagtttga | | NAHHLDHKMEIKQWKK | |
| | cccggaaattatcggtccacgggatattatcaaaattattgag | | SFLCSLVFGIPVMALMIY | |
| | gaaattggctttcatgcttccctggcccagagaaaccccaacg | | MLIPSNEPHQSMVLDH | |
| | ctcatcacttggaccacaagatggaaataaagcagtggaag | | NIIPGLSILNLIFFILCTFV | |
| | aagtctttcctgtgcagcctggtgtttggcatccctgtcatggcc | | QLLGGWYFYVQAYKSLR | |
| | ttaatgatctatatgctgatacccagcaacgagccccaccagtc | | HRSANMDVLIVLATSIAY | |
| | catggtcctggaccacaacatcattccaggactgtccattctaa | | VYSLVILVVAVAEKAERS | |
| | atctcatcttctttatcttgtgtacctttgtccagctcctcggtggg | | PVTFFDTPPMLFVFIALG | |
| | tggtacttctacgttcaggcctacaaatctctgagacacaggtc | | RWLEHLAKSKTSEALAKL | |
| | agccaacatggacgtgctcatcgtcctggccacaagcattgct | | MSLQATEATVVTLGEDN | |
| | tatgtttattctctggtcatcctggtggttgctgtggctgagaag | | LIIREEQVPMELVQRGDI | |
| | gcggagaggagccctgtgacattcttcgacacgccccccatg | | VKVVPGGKFPVDGKVLE | |
| | ctctttgtgttcattgccctgggccggtggctggaacacttggc | | GNTMADESLITGEAMPV | |
| | aaagagcaaaacctcagaagccctggctaaactcatgtctctc | | TKKPGSTVIAGSINAHGS | |
| | caagccacagaagccaccgttgtgacccttggtgaggacaat | | VLIKATHVGNDTTLAQIV | |
| | ttaatcatcagggaggagcaagtccccatggagctggtgca | | KLVEEAQMSKAPIQQLA | |
| | gcggggcgatatcgtcaaggtggtccctgggggaaagtttc | | DRFSGYFVPFIIIMSTLTL | |
| | cagtggatgggaaagtcctggaaggcaataccatggctgat | | VVWIVIGFIDFGVVQRYF | |
| | gagtccctcatcacaggagaagccatgccagtcactaagaaa | | PNPNKHISQTEVIIRFAF | |
| | cccggaagcactgtaattgcggggtctataaatgcacatggc | | QTSITVLCIACPCSLGLAT | |
| | tctgtgctcattaaagctacccacgtgggcaatgacaccacttt | | PTAVMVGTGVAAQNGI | |
| | ggctcagattgtgaaactggtggaagaggctcagatgtcaa | | LIKGGKPLEMAHKIKTV | |
| | aggcacccattcagcagctggctgaccggtttagtggatatttt | | MFDKTGTITHGVPRVM | |
| | gtcccatttatcatcatcatgtcaactttgacgttggtggtatgg | | RVLLLGDVATLPLRKVLA | |
| | attgtaatcggttttatcgattttggtgttgttcagagatactttcc | | VVGTAEASSEHPLGVAV | |
| | taaccccaacaagcacatctcccagacagaggtgatcatccg | | TKYCKEELGTETLGYCTD | |
| | gtttgctttccagacgtccatcacggtgctgtgcattgcctgccc | | FQAVPGCGIGCKVSNVE | |
| | ctgctccctggggctggccacgcccacggctgtcatggtggg | | GILAHSERPLSAPASHLN | |
| | caccggggggccgcgcagaacggcatcctcatcaaggga | | EAGSLPAEKDAVPQTFS | |
| | ggcaagcccctggagatggcgcacaagataaagactgtgat | | VLIGNREWLRRNGLTISS | |
| | gtttgacaagactggcaccattacccatggcgtccccagggtc | | DVSDAMTDHEMKGQT | |
| | atgcgggtgctcctgctgggggatgtggccacactgcccctc | | AILVAIDGVLCGMIAIAD | |
| | aggaaggttctggctgtggtggggactgcggaggccagca | | AVKQEAALAVHTLQSM | |
| | gtgaacaccccttgggcgtggcagtcaccaaatactgtaaag | | GVDVVLITGDNRKTARA | |
| | aggaacttggaacagagaccttgggatactgcacggacttcc | | IATQVGINKVFAEVLPSH | |
| | aggcagtgccaggctgtggaattgggtgcaaagtcagcaac | | KVAKVQELQNKGKKVA | |
| | gtggaaggcatcctggcccacagtgagcgccctttgagtgca | | MVGDGVNDSPALAQA | |
| | ccggccagtcacctgaatgaggctggcagccttcccgcaga | | DMGVAIGTGTDVAIEAA | |
| | aaaagatgcagtcccccagaccttctctgtgctgattggaaac | | DVVLIRNDLLDVVASIHL | |
| | cgtgagtggctgaggcgcaacggtttaaccatttctagcgat | | SKRTVRRIRINLVLALIYN | |
| | gtcagtgacgctatgacagaccacgagatgaaaggacagac | | LVGIPIAAGVFMPIGIVL | |
| | agccatcctggtggctattgacggtgtgctctgtgggatgatc | | QPWMGSAAMAASSVS | |
| | gcaatcgcagacgctgtcaagcaggaggctgccctggctgt | | VVLSSLQLKCYKKPDLER | |
| | gcacacgctgcagagcatgggtgtggacgtggttctgatcac | | YEAQAHGHMKPLTASQ | |
| | gggggacaaccggaagacagccagagctattgccacccag | | VSVHIGMDDRWRDSPR | |
| | gttggcatcaacaaagtctttgcagaggtgctgccttcgcaca | | ATPWDQVSYVSQVSLS | |
| | aggtggccaaggtccaggagctccagaataaagggaagaa | | SLTSDKPSRHSAAADDD | |
| | agtcgccatggtgggggatggggtcaatgactccccggcctt | | GDKWSLLLNGRDEEQYI | |
| | ggcccaggcagacatgggtgtggccattggcaccggcacg | | * | |
| | gatgtggccatcgaggcagccgacgtcgtccttatcagaaat | | | |
| | gatttgctggatgtggtggctagcattcacctttccaagagga | | | |
| | ctgtccgaaggatacgcatcaacctggtcctggcactgatttat | | | |
| | aacctggttgggatacccattgcagcaggtgtcttcatgcccat | | | |
| | cggcattgtgctgcagccctggatgggctcagcggccatgg | | | |
| | cagcctcctctgtgtctgtggtgctctcatccctgcagctcaagt | | | |
| | gctataagaagcctgacctggagaggtatgaggcacaggc | | | |
| | gcatggccacatgaagcccctgacggcatcccaggtcagtgt | | | |
| | gcacataggcatggatgacaggtggcgggactcccccagg | | | |
| | gccacaccatgggaccaggtcagctatgtcagccaggtgtc | | | |
| | gctgtcctccctgacgtccgacaagccatctcggcacagcgct | | | |
| | gcagcagacgatgatggggacaagtggtctctgctcctgaat | | | |
| | ggcagggatgaggagcagtacatctga | | | |
| Truncated | atgcctgagcaggagagacagatcacagccagagaaggg | 27 | MPEQERQITAREGASRKI | 28 |
| ATP7B | gccagtcggaaaatcttatctaagctttctttgcctacccgtgcc | | LSKLSLPTRAWEPAMKK | |
| (CRE2, | tgggaaccagcaatgaagaagagttttgcttttgacaatgttg | | SFAFDNVGYEGGLDGLG | |
| CRE5, and | gctatgaaggtggtctggatggcctgggcccttcttctcaggt | | PSSQVATSTVRVVKLRV | |
| CRE6 are | ggccaccagcacagtcagggtggtcaagctccgggtggag | | EGMTCQSCVSSIEGKVR | |
| present) | ggcatgacctgccagtcctgtgtcagctccattgaaggcaag | | KLQGVVRVKVSLSNQEA | |
| | gtccggaaactgcaaggagtagtgagagtcaaagtctcactc | | VITYQPYLIQPEDLRDHV | |
| | agcaaccaagaggccgtcatcacttatcagccttatctcattca | | NDMGFEAAIVSESCSTN | |
| | gcccgaagacctcagggaccatgtaaatgacatgggatttga | | PLGNHSAGNSMVQTTD | |
| | agctgccatcgtttctgaaagctgttctactaaccctcttggaaa | | GTPTSVQEVAPHTGRLP | |
| | ccacagtgctgggaattccatggtgcaaactacagatggtac | | ANHAPDILAKSPQSTRA | |
| | acctacatctgtgcaggaagtggctccccacactgggaggct | | VAPQKCFLQIKGMTCAS | |
| | ccctgcaaaccatgccccggacatcttggcaaagtccccaca | | CVSNIERNLQKEAGVLS | |
| | atcaaccagagcagtggcaccgcagaagtgcttcttacagat | | VLVALMAGKAEIKYDPE | |
| | caaaggcatgacctgtgcatcctgtgtgtctaacatagaaag | | VIQPLEIAQFIQDLGFEA | |
| | gaatctgcagaaagaagctggtgttctctccgtgttggttgcct | | AVMEDYAGSDGNIELTI | |
| | tgatggcaggaaaggcagagatcaagtatgacccagaggt | | TGMTCASCVHNIESKLT | |
| | catccagcccctcgagatagctcagttcatccaggacctgggt | | RTNGITYASVALATSKAL | |
| | tttgaggcagcagtcatggaggactacgcaggctccgatgg | | VKFDPEIIGPRDIIKIIEEIG | |
| | caacattgagctgacaatcacagggatgacctgcgcgtcctg | | FHASLAQRNPNAHHLD | |
| | tgtccacaacatagagtccaaactcacgaggacaaatggcat | | HKMEIKQWKKSFLCSLV | |
| | cacttatgcctccgttgcccttgccaccagcaaagcccttgttaa | | FGIPVMALMIYMLIPSNE | |
| | gtttgacccggaaattatcggtccacgggatattatcaaaatta | | PHQSMVLDHNIIPGLSIL | |
| | ttgaggaaattggctttcatgcttccctggcccagagaaaccc | | NLIFFILCTFVQLLGGWY | |
| | caacgctcatcacttggaccacaagatggaaataaagcagtg | | FYVQAYKSLRHRSANM | |
| | gaagaagtctttcctgtgcagcctggtgtttggcatccctgtca | | DVLIVLATSIAYVYSLVIL | |
| | tggccttaatgatctatatgctgatacccagcaacgagcccca | | VVAVAEKAERSPVTFFD | |
| | ccagtccatggtcctggaccacaacatcattccaggactgtcc | | TPPMLFVFIALGRWLEHL | |
| | attctaaatctcatcttctttatcttgtgtacctttgtccagctcc | | AKSKTSEALAKLMSLQA | |
| | tcggtgggtggtacttctacgttcaggcctacaaatctctgagac | | TEATVVTLGEDNLIIREE | |
| | acaggtcagccaacatggacgtgctcatcgtcctggccacaa | | QVPMELVQRGDIVKVV | |
| | gcattgcttatgtttattctctggtcatcctggtggttgctgtggc | | PGGKFPVDGKVLEGNT | |
| | tgagaaggcggagaggagccctgtgacattcttcgacacgc | | MADESLITGEAMPVTKK | |
| | cccccatgctctttgtgttcattgccctgggccggtggctggaa | | PGSTVIAGSINAHGSVLI | |
| | cacttggcaaagagcaaaacctcagaagccctggctaaactc | | KATHVGNDTTLAQIVKL | |
| | atgtctctccaagccacagaagccaccgttgtgacccttggtg | | VEEAQMSKAPIQQLAD | |
| | aggacaatttaatcatcagggaggagcaagtccccatggag | | RFSGYFVPFIIIMSTLTLVV | |
| | ctggtgcagcggggcgatatcgtcaaggtggtccctggggg | | WIVIGFIDFGVVQRYFPN | |
| | aaagtttccagtggatgggaaagtcctggaaggcaataccat | | PNKHISQTEVIIRFAFQTS | |
| | ggctgatgagtccctcatcacaggagaagccatgccagtcac | | ITVLCIACPCSLGLATPTA | |
| | taagaaacccggaagcactgtaattgcggggtctataaatgc | | VMVGTGVAAQNGILIKG | |
| | acatggctctgtgctcattaaagctacccacgtgggcaatgac | | GKPLEMAHKIKTVMFDK | |
| | accactttggctcagattgtgaaactggtggaagaggctcag | | TGTITHGVPRVMRVLLL | |
| | atgtcaaaggcacccattcagcagctggctgaccggtttagtg | | GDVATLPLRKVLAVVGT | |
| | gatattttgtcccatttatcatcatcatgtcaactttgacgttggtg | | AEASSEHPLGVAVTKYC | |
| | gtatggattgtaatcggttttatcgattttggtgttgttcagagat | | KEELGTETLGYCTDFQA | |
| | actttcctaaccccaacaagcacatctcccagacagaggtgat | | VPGCGIGCKVSNVEGIL | |
| | catccggtttgctttccagacgtccatcacggtgctgtgcattg | | AHSERPLSAPASHLNEA | |
| | cctgcccctgctccctggggctggccacgcccacggctgtcat | | GSLPAEKDAVPQTFSVLI | |
| | ggtgggcaccggggggccgcgcagaacggcatcctcatc | | GNREWLRRNGLTISSDV | |
| | aagggaggcaagcccctggagatggcgcacaagataaag | | SDAMTDHEMKGQTAIL | |
| | actgtgatgtttgacaagactggcaccattacccatggcgtcc | | VAIDGVLCGMIAIADAV | |
| | ccagggtcatgcgggtgctcctgctgggggatgtggccaca | | KQEAALAVHTLQSMGV | |
| | ctgcccctcaggaaggttctggctgtggtggggactgcgga | | DVVLITGDNRKTARAIAT | |
| | ggccagcagtgaacaccccttgggcgtggcagtcaccaaat | | QVGINKVFAEVLPSHKV | |
| | actgtaaagaggaacttggaacagagaccttgggatactgc | | AKVQELQNKGKKVAMV | |
| | acggacttccaggcagtgccaggctgtggaattgggtgcaa | | GDGVNDSPALAQADM | |
| | agtcagcaacgtggaaggcatcctggcccacagtgagcgcc | | GVAIGTGTDVAIEAADV | |
| | ctttgagtgcaccggccagtcacctgaatgaggctggcagcc | | VLIRNDLLDVVASIHLSK | |
| | ttcccgcagaaaaagatgcagtcccccagaccttctctgtgct | | RTVRRIRINLVLALIYNLV | |
| | gattggaaaccgtgagtggctgaggcgcaacggtttaaccat | | GIPIAAGVFMPIGIVLQP | |
| | ttctagcgatgtcagtgacgctatgacagaccacgagatgaa | | WMGSAAMAASSVSVV | |
| | aggacagacagccatcctggtggctattgacggtgtgctctgt | | LSSLQLKCYKKPDLERYE | |
| | gggatgatcgcaatcgcagacgctgtcaagcaggaggctgc | | AQAHGHMKPLTASQVS | |
| | cctggctgtgcacacgctgcagagcatgggtgtggacgtgg | | VHIGMDDRWRDSPRAT | |
| | ttctgatcacgggggacaaccggaagacagccagagctatt | | PWDQVSYVSQVSLSSLT | |
| | gccacccaggttggcatcaacaaagtctttgcagaggtgctg | | SDKPSRHSAAADDDGD | |
| | ccttcgcacaaggtggccaaggtccaggagctccagaataa | | KWSLLLNGRDEEQYI* | |
| | agggaagaaagtcgccatggtgggggatggggtcaatgac | | | |
| | tccccggccttggcccaggcagacatgggtgtggccattggc | | | |
| | accggcacggatgtggccatcgaggcagccgacgtcgtcct | | | |
| | tatcagaaatgatttgctggatgtggtggctagcattcacctttc | | | |
| | caagaggactgtccgaaggatacgcatcaacctggtcctggc | | | |
| | actgatttataacctggttgggatacccattgcagcaggtgtct | | | |
| | tcatgcccatcggcattgtgctgcagccctggatgggctcagc | | | |
| | ggccatggcagcctcctctgtgtctgtggtgctctcatccctgc | | | |
| | agctcaagtgctataagaagcctgacctggagaggtatgag | | | |
| | gcacaggcgcatggccacatgaagcccctgacggcatccca | | | |
| | ggtcagtgtgcacataggcatggatgacaggtggcgggact | | | |
| | cccccagggccacaccatgggaccaggtcagctatgtcagc | | | |
| | caggtgtcgctgtcctccctgacgtccgacaagccatctcggc | | | |
| | acagcgctgcagcagacgatgatggggacaagtggtctctg | | | |
| | ctcctgaatggcagggatgaggagcagtacatctga | | | |
| Truncated | atgcctgagcaggagagacagatcacagccagagaaggg | 29 | MPEQERQITAREGASRKI | 30 |
| ATP7B | gccagtcggaaaatcttatctaagctttctttgcctacccgtgcc | | LSKLSLPTRAWEPAMKK | |
| (CRE1, | tgggaaccagcaatgaagaagagttttgcttttgacaatgttg | | SFAFDNVGYEGGLDGLG | |
| CRE2, and | gctatgaaggtggtctggatggcctgggcccttcttctcaggt | | PSSQVATSTVRILGMTC | |
| CRE5 are | ggccaccagcacagtcaggatcttgggcatgacttgccagtc | | QSCVKSIEDRISNLKGIIS | |
| present) | atgtgtgaagtccattgaggacaggatttccaatttgaaaggc | | MKVSLEQGSATVKYVPS | |
| | atcatcagcatgaaggtttccctggaacaaggcagtgccact | | VVCLQQVCHQIGDMGF | |
| | gtgaaatatgtgccatcggttgtgtgcctgcaacaggtttgcc | | EASIAEGKAASWPSRSLP | |
| | atcaaattggggacatgggcttcgaggccagcattgcagaa | | AQEAVVKLRVEGMTCQ | |
| | ggaaaggcagcctcctggccctcaaggtccttgcctgcccag | | SCVSSIEGKVRKLQGVVR | |
| | gaggctgtggtcaagctccgggtggagggcatgacctgcca | | VKVSLSNQEAVITYQPYL | |
| | gtcctgtgtcagctccattgaaggcaaggtccggaaactgca | | IQPEDLRDHVNDMGFE | |
| | aggagtagtgagagtcaaagtctcactcagcaaccaagagg | | AAIVSESCSTNPLGNHS | |
| | ccgtcatcacttatcagccttatctcattcagcccgaagacctca | | AGNSMVQTTDGTPTSV | |
| | gggaccatgtaaatgacatgggatttgaagctgccatcgtttc | | QEVAPHTGRLPANHAP | |
| | tgaaagctgttctactaaccctcttggaaaccacagtgctggg | | DILAKSPQSTRAVAPQK | |
| | aattccatggtgcaaactacagatggtacacctacatctgtgc | | CFLQIKGMTCASCVSNIE | |
| | aggaagtggctccccacactgggaggctccctgcaaaccat | | RNLQKEAGVLSVLVALM | |
| | gccccggacatcttggcaaagtccccacaatcaaccagagca | | AGKAEIKYDPEVIQPLEIA | |
| | gtggcaccgcagaagtgcttcttacagatcaaaggcatgacc | | QFIQDLGFEAAVAQRNP | |
| | tgtgcatcctgtgtgtctaacatagaaaggaatctgcagaaag | | NAHHLDHKMEIKQWKK | |
| | aagctggtgttctctccgtgttggttgccttgatggcaggaaa | | SFLCSLVFGIPVMALMIY | |
| | ggcagagatcaagtatgacccagaggtcatccagcccctcg | | MLIPSNEPHQSMVLDH | |
| | agatagctcagttcatccaggacctgggttttgaggcagcagt | | NIIPGLSILNLIFFILCTFV | |
| | cgcccagagaaaccccaacgctcatcacttggaccacaagat | | QLLGGWYFYVQAYKSLR | |
| | ggaaataaagcagtggaagaagtctttcctgtgcagcctggt | | HRSANMDVLIVLATSIAY | |
| | gtttggcatccctgtcatggccttaatgatctatatgctgatacc | | VYSLVILVVAVAEKAERS | |
| | cagcaacgagccccaccagtccatggtcctggaccacaacat | | PVTFFDTPPMLFVFIALG | |
| | cattccaggactgtccattctaaatctcatcttctttatcttgtgta | | RWLEHLAKSKTSEALAKL | |
| | cctttgtccagctcctcggtgggtggtacttctacgttcaggcct | | MSLQATEATVVTLGEDN | |
| | acaaatctctgagacacaggtcagccaacatggacgtgctca | | LIIREEQVPMELVQRGDI | |
| | tcgtcctggccacaagcattgcttatgtttattctctggtcatcct | | VKVVPGGKFPVDGKVLE | |
| | ggtggttgctgtggctgagaaggcggagaggagccctgtg | | GNTMADESLITGEAMPV | |
| | acattcttcgacacgccccccatgctctttgtgttcattgccctgg | | TKKPGSTVIAGSINAHGS | |
| | gccggtggctggaacacttggcaaagagcaaaacctcagaa | | VLIKATHVGNDTTLAQIV | |
| | gccctggctaaactcatgtctctccaagccacagaagccaccg | | KLVEEAQMSKAPIQQLA | |
| | ttgtgacccttggtgaggacaatttaatcatcagggaggagc | | DRFSGYFVPFIIIMSTLTL | |
| | aagtccccatggagctggtgcagcggggcgatatcgtcaag | | VVWIVIGFIDFGVVQRYF | |
| | gtggtccctgggggaaagtttccagtggatgggaaagtcct | | PNPNKHISQTEVIIRFAF | |
| | ggaaggcaataccatggctgatgagtccctcatcacaggag | | QTSITVLCIACPCSLGLAT | |
| | aagccatgccagtcactaagaaacccggaagcactgtaattg | | PTAVMVGTGVAAQNGI | |
| | cggggtctataaatgcacatggctctgtgctcattaaagctacc | | LIKGGKPLEMAHKIKTV | |
| | cacgtgggcaatgacaccactttggctcagattgtgaaactg | | MFDKTGTITHGVPRVM | |
| | gtggaagaggctcagatgtcaaaggcacccattcagcagct | | RVLLLGDVATLPLRKVLA | |
| | ggctgaccggtttagtggatattttgtcccatttatcatcatcatg | | VVGTAEASSEHPLGVAV | |
| | tcaactttgacgttggtggtatggattgtaatcggttttatcgatt | | TKYCKEELGTETLGYCTD | |
| | ttggtgttgttcagagatactttcctaaccccaacaagcacatct | | FQAVPGCGIGCKVSNVE | |
| | cccagacagaggtgatcatccggtttgctttccagacgtccat | | GILAHSERPLSAPASHLN | |
| | cacggtgctgtgcattgcctgcccctgctccctggggctggcc | | EAGSLPAEKDAVPQTFS | |
| | acgcccacggctgtcatggtgggcaccggggtggccgcgc | | VLIGNREWLRRNGLTISS | |
| | agaacggcatcctcatcaagggaggcaagcccctggagatg | | DVSDAMTDHEMKGQT | |
| | gcgcacaagataaagactgtgatgtttgacaagactggcacc | | AILVAIDGVLCGMIAIAD | |
| | attacccatggcgtccccagggtcatgcgggtgctcctgctgg | | AVKQEAALAVHTLQSM | |
| | gggatgtggccacactgcccctcaggaaggttctggctgtgg | | GVDVVLITGDNRKTARA | |
| | tggggactgcggaggccagcagtgaacaccccttgggcgt | | IATQVGINKVFAEVLPSH | |
| | ggcagtcaccaaatactgtaaagaggaacttggaacagaga | | KVAKVQELQNKGKKVA | |
| | ccttgggatactgcacggacttccaggcagtgccaggctgtg | | MVGDGVNDSPALAQA | |
| | gaattgggtgcaaagtcagcaacgtggaaggcatcctggcc | | DMGVAIGTGTDVAIEAA | |
| | cacagtgagcgccctttgagtgcaccggccagtcacctgaat | | DVVLIRNDLLDVVASIHL | |
| | gaggctggcagccttcccgcagaaaaagatgcagtccccca | | SKRTVRRIRINLVLALIYN | |
| | gaccttctctgtgctgattggaaaccgtgagtggctgaggcg | | LVGIPIAAGVFMPIGIVL | |
| | caacggtttaaccatttctagcgatgtcagtgacgctatgaca | | QPWMGSAAMAASSVS | |
| | gaccacgagatgaaaggacagacagccatcctggtggctat | | VVLSSLQLKCYKKPDLER | |
| | tgacggtgtgctctgtgggatgatcgcaatcgcagacgctgt | | YEAQAHGHMKPLTASQ | |
| | caagcaggaggctgccctggctgtgcacacgctgcagagca | | VSVHIGMDDRWRDSPR | |
| | tgggtgtggacgtggttctgatcacgggggacaaccggaag | | ATPWDQVSYVSQVSLS | |
| | acagccagagctattgccacccaggttggcatcaacaaagtc | | SLTSDKPSRHSAAADDD | |
| | tttgcagaggtgctgccttcgcacaaggtggccaaggtccag | | GDKWSLLLNGRDEEQYI | |
| | gagctccagaataaagggaagaaagtcgccatggtggggg | | * | |
| | atggggtcaatgactccccggccttggcccaggcagacatg | | | |
| | ggtgtggccattggcaccggcacggatgtggccatcgaggc | | | |
| | agccgacgtcgtccttatcagaaatgatttgctggatgtggtg | | | |
| | gctagcattcacctttccaagaggactgtccgaaggatacgca | | | |
| | tcaacctggtcctggcactgatttataacctggttgggataccc | | | |
| | attgcagcaggtgtcttcatgcccatcggcattgtgctgcagc | | | |
| | cctggatgggctcagcggccatggcagcctcctctgtgtctgt | | | |
| | ggtgctctcatccctgcagctcaagtgctataagaagcctgac | | | |
| | ctggagaggtatgaggcacaggcgcatggccacatgaagc | | | |
| | ccctgacggcatcccaggtcagtgtgcacataggcatggatg | | | |
| | acaggtggcgggactcccccagggccacaccatgggacca | | | |
| | ggtcagctatgtcagccaggtgtcgctgtcctccctgacgtcc | | | |
| | gacaagccatctcggcacagcgctgcagcagacgatgatgg | | | |
| | ggacaagtggtctctgctcctgaatggcagggatgaggagc | | | |
| | agtacatctga | | | |
| Truncated | atgcctgagcaggagagacagatcacagccagagaaggg | 31 | MPEQERQITAREGASRKI | 32 |
| ATP7B | gccagtcggaaaatcttatctaagctttctttgcctacccgtgcc | | LSKLSLPTRAWEPAMKK | |
| (CRE1, | tgggaaccagcaatgaagaagagttttgcttttgacaatgttg | | SFAFDNVGYEGGLDGLG | |
| CRE2, and | gctatgaaggtggtctggatggcctgggcccttcttctcaggt | | PSSQVATSTVRILGMTC | |
| CRE6 are | ggccaccagcacagtcaggatcttgggcatgacttgccagtc | | QSCVKSIEDRISNLKGIIS | |
| present) | atgtgtgaagtccattgaggacaggatttccaatttgaaaggc | | MKVSLEQGSATVKYVPS | |
| | atcatcagcatgaaggtttccctggaacaaggcagtgccact | | VVCLQQVCHQIGDMGF | |
| | gtgaaatatgtgccatcggttgtgtgcctgcaacaggtttgcc | | EASIAEGKAASWPSRSLP | |
| | atcaaattggggacatgggcttcgaggccagcattgcagaa | | AQEAVVKLRVEGMTCQ | |
| | ggaaaggcagcctcctggccctcaaggtccttgcctgcccag | | SCVSSIEGKVRKLQGVVR | |
| | gaggctgtggtcaagctccgggtggagggcatgacctgcca | | VKVSLSNQEAVITYQPYL | |
| | gtcctgtgtcagctccattgaaggcaaggtccggaaactgca | | IQPEDLRDHVNDMGFE | |
| | aggagtagtgagagtcaaagtctcactcagcaaccaagagg | | AAIMEDYAGSDGNIELTI | |
| | ccgtcatcacttatcagccttatctcattcagcccgaagacctca | | TGMTCASCVHNIESKLT | |
| | gggaccatgtaaatgacatgggatttgaagctgccatcatgg | | RTNGITYASVALATSKAL | |
| | aggactacgcaggctccgatggcaacattgagctgacaatca | | VKFDPEIIGPRDIIKIIEEIG | |
| | cagggatgacctgcgcgtcctgtgtccacaacatagagtcca | | FHASLAQRNPNAHHLD | |
| | aactcacgaggacaaatggcatcacttatgcctccgttgccctt | | HKMEIKQWKKSFLCSLV | |
| | gccaccagcaaagcccttgttaagtttgacccggaaattatcg | | FGIPVMALMIYMLIPSNE | |
| | gtccacgggatattatcaaaattattgaggaaattggctttcat | | PHQSMVLDHNIIPGLSIL | |
| | gcttccctggcccagagaaaccccaacgctcatcacttggacc | | NLIFFILCTFVQLLGGWY | |
| | acaagatggaaataaagcagtggaagaagtctttcctgtgca | | FYVQAYKSLRHRSANM | |
| | gcctggtgtttggcatccctgtcatggccttaatgatctatatgc | | DVLIVLATSIAYVYSLVIL | |
| | tgatacccagcaacgagccccaccagtccatggtcctggacc | | VVAVAEKAERSPVTFFD | |
| | acaacatcattccaggactgtccattctaaatctcatcttctttatc | | TPPMLFVFIALGRWLEHL | |
| | ttgtgtacctttgtccagctcctcggtgggtggtacttctacgttc | | AKSKTSEALAKLMSLQA | |
| | aggcctacaaatctctgagacacaggtcagccaacatggac | | TEATVVTLGEDNLIIREE | |
| | gtgctcatcgtcctggccacaagcattgcttatgtttattctctg | | QVPMELVQRGDIVKVV | |
| | gtcatcctggtggttgctgtggctgagaaggcggagaggag | | PGGKFPVDGKVLEGNT | |
| | ccctgtgacattcttcgacacgccccccatgctctttgtgttcatt | | MADESLITGEAMPVTKK | |
| | gccctgggccggtggctggaacacttggcaaagagcaaaa | | PGSTVIAGSINAHGSVLI | |
| | cctcagaagccctggctaaactcatgtctctccaagccacaga | | KATHVGNDTTLAQIVKL | |
| | agccaccgttgtgacccttggtgaggacaatttaatcatcagg | | VEEAQMSKAPIQQLAD | |
| | gaggagcaagtccccatggagctggtgcagcggggcgata | | RFSGYFVPFIIIMSTLTLVV | |
| | tcgtcaaggtggtccctgggggaaagtttccagtggatggg | | WIVIGFIDFGVVQRYFPN | |
| | aaagtcctggaaggcaataccatggctgatgagtccctcatc | | PNKHISQTEVIIRFAFQTS | |
| | acaggagaagccatgccagtcactaagaaacccggaagca | | ITVLCIACPCSLGLATPTA | |
| | ctgtaattgcggggtctataaatgcacatggctctgtgctcatt | | VMVGTGVAAQNGILIKG | |
| | aaagctacccacgtgggcaatgacaccactttggctcagattg | | GKPLEMAHKIKTVMFDK | |
| | tgaaactggtggaagaggctcagatgtcaaaggcacccattc | | TGTITHGVPRVMRVLLL | |
| | agcagctggctgaccggtttagtggatattttgtcccatttatca | | GDVATLPLRKVLAVVGT | |
| | tcatcatgtcaactttgacgttggtggtatggattgtaatcggtt | | AEASSEHPLGVAVTKYC | |
| | ttatcgattttggtgttgttcagagatactttcctaaccccaacaa | | KEELGTETLGYCTDFQA | |
| | gcacatctcccagacagaggtgatcatccggtttgctttccag | | VPGCGIGCKVSNVEGIL | |
| | acgtccatcacggtgctgtgcattgcctgcccctgctccctgg | | AHSERPLSAPASHLNEA | |
| | ggctggccacgcccacggctgtcatggtgggcaccggggt | | GSLPAEKDAVPQTFSVLI | |
| | ggccgcgcagaacggcatcctcatcaagggaggcaagccc | | GNREWLRRNGLTISSDV | |
| | ctggagatggcgcacaagataaagactgtgatgtttgacaag | | SDAMTDHEMKGQTAIL | |
| | actggcaccattacccatggcgtccccagggtcatgcgggtg | | VAIDGVLCGMIAIADAV | |
| | ctcctgctgggggatgtggccacactgcccctcaggaaggtt | | KQEAALAVHTLQSMGV | |
| | ctggctgtggtggggactgcggaggccagcagtgaacacc | | DVVLITGDNRKTARAIAT | |
| | ccttgggcgtggcagtcaccaaatactgtaaagaggaacttg | | QVGINKVFAEVLPSHKV | |
| | gaacagagaccttgggatactgcacggacttccaggcagtg | | AKVQELQNKGKKVAMV | |
| | ccaggctgtggaattgggtgcaaagtcagcaacgtggaag | | GDGVNDSPALAQADM | |
| | gcatcctggcccacagtgagcgccctttgagtgcaccggcca | | GVAIGTGTDVAIEAADV | |
| | gtcacctgaatgaggctggcagccttcccgcagaaaaagat | | VLIRNDLLDVVASIHLSK | |
| | gcagtcccccagaccttctctgtgctgattggaaaccgtgagt | | RTVRRIRINLVLALIYNLV | |
| | ggctgaggcgcaacggtttaaccatttctagcgatgtcagtga | | GIPIAAGVFMPIGIVLQP | |
| | cgctatgacagaccacgagatgaaaggacagacagccatcc | | WMGSAAMAASSVSVV | |
| | tggtggctattgacggtgtgctctgtgggatgatcgcaatcgc | | LSSLQLKCYKKPDLERYE | |
| | agacgctgtcaagcaggaggctgccctggctgtgcacacgc | | AQAHGHMKPLTASQVS | |
| | tgcagagcatgggtgtggacgtggttctgatcacgggggac | | VHIGMDDRWRDSPRAT | |
| | aaccggaagacagccagagctattgccacccaggttggcat | | PWDQVSYVSQVSLSSLT | |
| | caacaaagtctttgcagaggtgctgccttcgcacaaggtggc | | SDKPSRHSAAADDDGD | |
| | caaggtccaggagctccagaataaagggaagaaagtcgcc | | KWSLLLNGRDEEQYI* | |
| | atggtgggggatggggtcaatgactccccggccttggccca | | | |
| | ggcagacatgggtgtggccattggcaccggcacggatgtg | | | |
| | gccatcgaggcagccgacgtcgtccttatcagaaatgatttgc | | | |
| | tggatgtggtggctagcattcacctttccaagaggactgtccg | | | |
| | aaggatacgcatcaacctggtcctggcactgatttataacctg | | | |
| | gttgggatacccattgcagcaggtgtcttcatgcccatcggca | | | |
| | ttgtgctgcagccctggatgggctcagcggccatggcagcct | | | |
| | cctctgtgtctgtggtgctctcatccctgcagctcaagtgctata | | | |
| | agaagcctgacctggagaggtatgaggcacaggcgcatgg | | | |
| | ccacatgaagcccctgacggcatcccaggtcagtgtgcacat | | | |
| | aggcatggatgacaggtggcgggactcccccagggccaca | | | |
| | ccatgggaccaggtcagctatgtcagccaggtgtcgctgtcct | | | |
| | ccctgacgtccgacaagccatctcggcacagcgctgcagca | | | |
| | gacgatgatggggacaagtggtctctgctcctgaatggcag | | | |
| | ggatgaggagcagtacatctga | | | |
| Truncated | atgcctgagcaggagagacagatcacagccagagaaggg | 33 | MPEQERQITAREGASRKI | 34 |
| ATP7B | gccagtcggaaaatcttatctaagctttctttgcctacccgtgcc | | LSKLSLPTRAWEPAMKK | |
| (CRE1, | tgggaaccagcaatgaagaagagttttgcttttgacaatgttg | | SFAFDNVGYEGGLDGLG | |
| CRE2, | gctatgaaggtggtctggatggcctgggcccttcttctcaggt | | PSSQVATSTVRILGMTC | |
| CRE5, and | ggccaccagcacagtcaggatcttgggcatgacttgccagtc | | QSCVKSIEDRISNLKGIIS | |
| CRE6 are | atgtgtgaagtccattgaggacaggatttccaatttgaaaggc | | MKVSLEQGSATVKYVPS | |
| present) | atcatcagcatgaaggtttccctggaacaaggcagtgccact | | VVCLQQVCHQIGDMGF | |
| | gtgaaatatgtgccatcggttgtgtgcctgcaacaggtttgcc | | EASIAEGKAASWPSRSLP | |
| | atcaaattggggacatgggcttcgaggccagcattgcagaa | | AQEAVVKLRVEGMTCQ | |
| | ggaaaggcagcctcctggccctcaaggtccttgcctgcccag | | SCVSSIEGKVRKLQGVVR | |
| | gaggctgtggtcaagctccgggtggagggcatgacctgcca | | VKVSLSNQEAVITYQPYL | |
| | gtcctgtgtcagctccattgaaggcaaggtccggaaactgca | | IQPEDLRDHVNDMGFE | |
| | aggagtagtgagagtcaaagtctcactcagcaaccaagagg | | AAIVSESCSTNPLGNHS | |
| | ccgtcatcacttatcagccttatctcattcagcccgaagacctca | | AGNSMVQTTDGTPTSV | |
| | gggaccatgtaaatgacatgggatttgaagctgccatcgtttc | | QEVAPHTGRLPANHAP | |
| | tgaaagctgttctactaaccctcttggaaaccacagtgctggg | | DILAKSPQSTRAVAPQK | |
| | aattccatggtgcaaactacagatggtacacctacatctgtgc | | CFLQIKGMTCASCVSNIE | |
| | aggaagtggctccccacactgggaggctccctgcaaaccat | | RNLQKEAGVLSVLVALM | |
| | gccccggacatcttggcaaagtccccacaatcaaccagagca | | AGKAEIKYDPEVIQPLEIA | |
| | gtggcaccgcagaagtgcttcttacagatcaaaggcatgacc | | QFIQDLGFEAAVMEDYA | |
| | tgtgcatcctgtgtgtctaacatagaaaggaatctgcagaaag | | GSDGNIELTITGMTCAS | |
| | aagctggtgttctctccgtgttggttgccttgatggcaggaaa | | CVHNIESKLTRTNGITYA | |
| | ggcagagatcaagtatgacccagaggtcatccagcccctcg | | SVALATSKALVKFDPEIIG | |
| | agatagctcagttcatccaggacctgggttttgaggcagcagt | | PRDIIKIIEEIGFHASLAQR | |
| | catggaggactacgcaggctccgatggcaacattgagctga | | NPNAHHLDHKMEIKQ | |
| | caatcacagggatgacctgcgcgtcctgtgtccacaacatag | | WKKSFLCSLVFGIPVMAL | |
| | agtccaaactcacgaggacaaatggcatcacttatgcctccgt | | MIYMLIPSNEPHQSMVL | |
| | tgcccttgccaccagcaaagcccttgttaagtttgacccggaa | | DHNIIPGLSILNLIFFILCT | |
| | attatcggtccacgggatattatcaaaattattgaggaaattgg | | FVQLLGGWYFYVQAYK | |
| | ctttcatgcttccctggcccagagaaaccccaacgctcatcact | | SLRHRSANMDVLIVLAT | |
| | tggaccacaagatggaaataaagcagtggaagaagtctttcc | | SIAYVYSLVILVVAVAEKA | |
| | tgtgcagcctggtgtttggcatccctgtcatggccttaatgatct | | ERSPVTFFDTPPMLFVFI | |
| | atatgctgatacccagcaacgagccccaccagtccatggtcct | | ALGRWLEHLAKSKTSEA | |
| | ggaccacaacatcattccaggactgtccattctaaatctcatctt | | LAKLMSLQATEATVVTL | |
| | ctttatcttgtgtacctttgtccagctcctcggtgggtggtacttc | | GEDNLIIREEQVPMELV | |
| | tacgttcaggcctacaaatctctgagacacaggtcagccaaca | | QRGDIVKVVPGGKFPVD | |
| | tggacgtgctcatcgtcctggccacaagcattgcttatgtttatt | | GKVLEGNTMADESLITG | |
| | ctctggtcatcctggtggttgctgtggctgagaaggcggaga | | EAMPVTKKPGSTVIAGSI | |
| | ggagccctgtgacattcttcgacacgccccccatgctctttgtg | | NAHGSVLIKATHVGNDT | |
| | ttcattgccctgggccggtggctggaacacttggcaaagagc | | TLAQIVKLVEEAQMSKA | |
| | aaaacctcagaagccctggctaaactcatgtctctccaagcca | | PIQQLADRFSGYFVPFIII | |
| | cagaagccaccgttgtgacccttggtgaggacaatttaatcat | | MSTLTLVVWIVIGFIDFG | |
| | cagggaggagcaagtccccatggagctggtgcagcgggg | | VVQRYFPNPNKHISQTE | |
| | cgatatcgtcaaggtggtccctgggggaaagtttccagtgga | | VIIRFAFQTSITVLCIACPC | |
| | tgggaaagtcctggaaggcaataccatggctgatgagtccct | | SLGLATPTAVMVGTGVA | |
| | catcacaggagaagccatgccagtcactaagaaacccggaa | | AQNGILIKGGKPLEMAH | |
| | gcactgtaattgcggggtctataaatgcacatggctctgtgctc | | KIKTVMFDKTGTITHGVP | |
| | attaaagctacccacgtgggcaatgacaccactttggctcaga | | RVMRVLLLGDVATLPLR | |
| | ttgtgaaactggtggaagaggctcagatgtcaaaggcaccc | | KVLAVVGTAEASSEHPL | |
| | attcagcagctggctgaccggtttagtggatattttgtcccattt | | GVAVTKYCKEELGTETLG | |
| | atcatcatcatgtcaactttgacgttggtggtatggattgtaatc | | YCTDFQAVPGCGIGCKV | |
| | ggttttatcgattttggtgttgttcagagatactttcctaacccca | | SNVEGILAHSERPLSAPA | |
| | acaagcacatctcccagacagaggtgatcatccggtttgctttc | | SHLNEAGSLPAEKDAVP | |
| | cagacgtccatcacggtgctgtgcattgcctgcccctgctccct | | QTFSVLIGNREWLRRNG | |
| | ggggctggccacgcccacggctgtcatggtgggcaccggg | | LTISSDVSDAMTDHEMK | |
| | gtggccgcgcagaacggcatcctcatcaagggaggcaagc | | GQTAILVAIDGVLCGMI | |
| | ccctggagatggcgcacaagataaagactgtgatgtttgaca | | AIADAVKQEAALAVHTL | |
| | agactggcaccattacccatggcgtccccagggtcatgcggg | | QSMGVDVVLITGDNRK | |
| | tgctcctgctgggggatgtggccacactgcccctcaggaagg | | TARAIATQVGINKVFAEV | |
| | ttctggctgtggtggggactgcggaggccagcagtgaacac | | LPSHKVAKVQELQNKGK | |
| | cccttgggcgtggcagtcaccaaatactgtaaagaggaactt | | KVAMVGDGVNDSPALA | |
| | ggaacagagaccttgggatactgcacggacttccaggcagt | | QADMGVAIGTGTDVAIE | |
| | gccaggctgtggaattgggtgcaaagtcagcaacgtggaa | | AADVVLIRNDLLDVVASI | |
| | ggcatcctggcccacagtgagcgccctttgagtgcaccggcc | | HLSKRTVRRIRINLVLALI | |
| | agtcacctgaatgaggctggcagccttcccgcagaaaaagat | | YNLVGIPIAAGVFMPIGI | |
| | gcagtcccccagaccttctctgtgctgattggaaaccgtgagt | | VLQPWMGSAAMAASS | |
| | ggctgaggcgcaacggtttaaccatttctagcgatgtcagtga | | VSVVLSSLQLKCYKKPDL | |
| | cgctatgacagaccacgagatgaaaggacagacagccatcc | | ERYEAQAHGHMKPLTA | |
| | tggtggctattgacggtgtgctctgtgggatgatcgcaatcgc | | SQVSVHIGMDDRWRDS | |
| | agacgctgtcaagcaggaggctgccctggctgtgcacacgc | | PRATPWDQVSYVSQVS | |
| | tgcagagcatgggtgtggacgtggttctgatcacgggggac | | LSSLTSDKPSRHSAAAD | |
| | aaccggaagacagccagagctattgccacccaggttggcat | | DDGDKWSLLLNGRDEE | |
| | caacaaagtctttgcagaggtgctgccttcgcacaaggtggc | | QYI* | |
| | caaggtccaggagctccagaataaagggaagaaagtcgcc | | | |
| | atggtgggggatggggtcaatgactccccggccttggccca | | | |
| | ggcagacatgggtgtggccattggcaccggcacggatgtg | | | |
| | gccatcgaggcagccgacgtcgtccttatcagaaatgatttgc | | | |
| | tggatgtggtggctagcattcacctttccaagaggactgtccg | | | |
| | aaggatacgcatcaacctggtcctggcactgatttataacctg | | | |
| | gttgggatacccattgcagcaggtgtcttcatgcccatcggca | | | |
| | ttgtgctgcagccctggatgggctcagcggccatggcagcct | | | |
| | cctctgtgtctgtggtgctctcatccctgcagctcaagtgctata | | | |
| | agaagcctgacctggagaggtatgaggcacaggcgcatgg | | | |
| | ccacatgaagcccctgacggcatcccaggtcagtgtgcacat | | | |
| | aggcatggatgacaggtggcgggactcccccagggccaca | | | |
| | ccatgggaccaggtcagctatgtcagccaggtgtcgctgtcct | | | |
| | ccctgacgtccgacaagccatctcggcacagcgctgcagca | | | |
| | gacgatgatggggacaagtggtctctgctcctgaatggcag | | | |
| | ggatgaggagcagtacatctga | | | |

In some embodiment, CRE3 has been deleted, but CRE5 or variant thereof and/or CRE6 or variant thereof was present, the said truncated ATP7B further comprises CRE4 or variant thereof.

In some embodiment, CRE3 has been deleted, but CRE5 or variant thereof and/or CRE6 or variant thereof was present, CRE1 or variant thereof and/or CRE2 or variant thereof was present, the said truncated ATP7B further comprises CRE4 or variant thereof.

In some embodiment, this disclosure provides a truncated ATP7B in which CRE1 or variant thereof, CRE4 or variant thereof, and CRE5 or variant thereof are present, but CRE2, CRE3 and CRE6 have been deleted. In some embodiment, this disclosure provides a truncated ATP7B in which CRE1 or variant thereof, CRE4 or variant thereof, and CRE6 or variant thereof are present, but CRE2, CRE3 and CRE5 have been deleted. In some embodiment, this disclosure provides a truncated ATP7B in which CRE1 or variant thereof, CRE4 or variant thereof, CRE5 or variant thereof, and CRE6 or variant thereof are present, but CRE2 and CRE3 have been deleted.

In some embodiment, this disclosure provides a truncated ATP7B in which CRE2 or variant thereof, CRE4 or variant thereof, and CRE5 or variant thereof are present, but CRE1, CRE3, and CRE6 have been deleted. In some embodiment, this disclosure provides a truncated ATP7B in which CRE2 or variant thereof, CRE4 or variant thereof, and CRE6 or variant thereof are present, but CRE1, CRE3, and CRE5 have been deleted. In some embodiment, this disclosure provides a truncated ATP7B in which CRE2 or variant thereof, CRE4 or variant thereof, CRE5 or variant thereof, and CRE6 or variant thereof are present, but CRE1 and CRE3 have been deleted.

In some embodiment, this disclosure provides a truncated ATP7B in which CRE1 or variant thereof, CRE2 or variant thereof, CRE4 or variant thereof, and CRE5 or variant thereof are present, but CRE3 and CRE6 have been deleted. In some embodiment, this disclosure provides a truncated ATP7B in which CRE1 or variant thereof, CRE2 or variant thereof, CRE4 or variant thereof, and CRE6 or variant thereof are present, but CRE3 and CRE5 have been deleted. In some embodiment, this disclosure provides a truncated ATP7B in which CRE1 or variant thereof, CRE2 or variant thereof, CRE4 or variant thereof, CRE5 or variant thereof, and CRE6 or variant thereof are present, but CRE3 has been deleted.

In some embodiment, CRE3 was not deleted in the truncated ATP7B. Preferably, this disclosure provides a truncated ATP7B in which CRE1 or variant thereof, CRE3 or variant thereof, and CRE5 or variant thereof are present, but CRE2, CRE4 and CRE6 have been deleted. Preferably, this disclosure provides a truncated ATP7B in which CRE1 or variant thereof, CRE3 or variant thereof, and CRE6 or variant thereof are present, but CRE2, CRE4, and CRE5 have been deleted. Preferably, this disclosure provides a truncated ATP7B in which CRE1 or variant thereof, CRE3 or variant thereof, CRE5 or variant thereof, and CRE6 or variant thereof are present, but CRE2 and CRE4 have been deleted. Preferably, this disclosure provides a truncated ATP7B in which CRE2 or variant thereof, CRE3 or variant thereof, and CRE5 or variant thereof are present, but CRE1, CRE4 and CRE6 have been deleted. Preferably, this disclosure provides a truncated ATP7B in which CRE2 or variant thereof, CRE3 or variant thereof, and CRE6 or variant thereof are present, but CRE1, CRE4, and CRE5 have been deleted. Preferably, this disclosure provides a truncated ATP7B in which CRE2 or variant thereof, CRE3 or variant thereof, CRE5 or variant thereof, and CRE6 or variant thereof are present, but CRE1 and CRE4 have been deleted. Preferably, this disclosure provides a truncated ATP7B in which CRE1 or variant thereof, CRE2 or variant thereof, CRE3 or variant thereof, and CRE5 or variant thereof are present, but CRE4, and CRE6 have been deleted. Preferably, this disclosure provides a truncated ATP7B in which CRE1 or variant thereof, CRE2 or variant thereof, CRE3 or variant thereof, and CRE6 or variant thereof are present, but CRE4 and CRE5 have been deleted. Preferably, this disclosure provides a truncated ATP7B in which CRE1 or variant thereof, CRE2 or variant thereof, CRE3 or variant thereof, CRE5 or variant thereof, and CRE6 or variant thereof are present, but CRE4 have been deleted.

In some embodiment, CRE3 has been deleted, but CRE5 or variant thereof and/or CRE6 or variant thereof was present, CRE1 or variant thereof and/or CRE2 or variant thereof was present, the said truncated ATP7B further comprises CRE4 or variant thereof. Preferably, this disclosure provides a truncated ATP7B in which CRE1 or variant thereof, CRE4 or variant thereof, and CRE5 or variant thereof are present, but CRE2, CRE3 and CRE6 have been deleted. Preferably, this disclosure provides a truncated ATP7B in which CRE1 or variant thereof, CRE4 or variant thereof, and CRE6 or variant thereof are present, but CRE2, CRE3, CRE5 have been deleted. Preferably, this disclosure provides a truncated ATP7B in which CRE1 or variant thereof, CRE4 or variant thereof, CRE5 or variant thereof, and CRE6 or variant thereof are present, but CRE2 and CRE3 have been deleted. Preferably, this disclosure provides a truncated ATP7B in which CRE2 or variant thereof, CRE4 or variant thereof, and CRE5 or variant thereof are present, but CRE1, CRE3 and CRE6 have been deleted. Preferably, this disclosure provides a truncated ATP7B in which CRE2 or variant thereof, CRE4 or variant thereof, and CRE6 or variant thereof are present, but CRE1, CRE3, and CRE5 have been deleted. Preferably, this disclosure provides a truncated ATP7B in which CRE2 or variant thereof, CRE4 or variant thereof, CRE5 or variant thereof, and CRE6 or variant thereof are present, but CRE1 and CRE3 have been deleted. Preferably, this disclosure provides a truncated ATP7B in which CRE1 or variant thereof, CRE2 or variant thereof, CRE4 or variant thereof, and CRE5 or variant thereof are present, but CRE3, and CRE6 have been deleted. Preferably, this disclosure provides a truncated ATP7B in which CRE1 or variant thereof, CRE2 or variant thereof, CRE4 or variant thereof, and CRE6 or variant thereof are present, but CRE3 and CRE5 have been deleted. Preferably, this disclosure provides a truncated ATP7B in which CRE1 or variant thereof, CRE2 or variant thereof, CRE4 or variant thereof, CRE5 or variant thereof, and CRE6 or variant thereof are present, but CRE3 have been deleted.

In another aspect, this disclosure provides a nucleic acid sequence encoding the said truncated ATP7B.

In some embodiment, truncated ATP7B nucleotide comprises a sequence selected from the group consisting of SEQ ID NOs: 17, 19, 21, 23, 25, 27, 29, 31, and 33, and at least 80%, at least 85%, at least 90%, at least 95%, at least 99%, at least 99.5%, at least 99.8% identical to SEQ ID NOs: 17, 19, 21, 23, 25, 27, 29, 31, and 33, wherein the truncated ATP7B nucleotide encodes the truncated ATP7B protein that retains the functionality of transmembrane copper transportation.

In another aspect, this disclosure provides encoding the truncated ATP7B of present disclosure. In some embodiment, the RNA comprises a messenger RNA (mRNA) or a circular RNA (circRNA).

### Recombinant Nucleic Acid Construct

In another aspect, this disclosure provides a recombinant nucleic acid construct comprising:
(a) a 5′-inverted terminal repeat (ITR) sequence;
(b) a promoter sequence;
(c) the said nucleic acid sequence encoding the said truncated ATP7B;
(d) a poly(A) sequence; and
(e) a 3′-ITR sequence.

In some embodiment, preferably, the promoter was selected, truncated or optimized from a transthyretin (TTR) promoter, a chicken b-actin (CBA) promoter, a cytomegalovirus immediate early gene (CMV) promoter, a thyroxine binding globulin (TBG) promoter, an alpha-1 anti-trypsin (A1AT) promoter, and a CAG promoter.

In some embodiment, the recombinant nucleic acid construct further comprises one or more enhancer sequences. The enhancer was preferably selected, truncated or optimized from a transthyretin enhancer (enTTR), a cytomegalovirus immediate early gene (CMV) enhancer, a chicken β-actin (CBA) enhancer, an En34 enhancer, and an apolipoprotein (ApoE) enhancer.

In some embodiment, the recombinant nucleic acid construct further comprises one or more intron sequences. The intron was preferably selected, truncated or optimized from endogenous intron from ATP7B, an SV40 Small T intron, a rabbit hemoglobin subunit beta (rHBB) intron, a human beta globin IV S2 intron, a Promega chimeric intron, and a hFIX intron.

In some embodiment, the recombinant nucleic acid construct further comprises one or more signal sequences. Signal sequence was preferably selected from a SV40 polyadenylation signal sequence, a bovine growth hormone (BGH) polyadenylation signal sequence, and a rabbit beta globin polyadenylation signal sequence.

### Recombinant Vector

In another aspect, this disclosure provides a vector comprising the said recombinant nucleic acid construct. preferably, the vector was a virus vector, preferably was AAV vector. The term “vector” was a nucleic acid molecule allowing insertion of foreign nucleic acid without disrupting the ability of the vector to retain, replicate and/or integrate in a host cell. A vector can include nucleic acid sequences that permit it to replicate in a host cell, such as an origin of replication. A vector can also include one or more selectable marker genes and other genetic elements. An expression vector was a vector that contains the necessary regulatory sequences to allow transcription and translation of inserted gene or genes. In some embodiments herein, the vector was an AAV vector.

In various embodiments described herein, the recombinant virus vector was an adeno-associated virus (AAV) vector. The AAV vector can be an AAV vector of serotype 1, 2, 3, 3B, 4, 5, 6, 7, 8, 9, 10, 11, 12 or 13, e.g., AAV1, AAV2, AAV3, AAV3B, AAV4, AAV5, AAV6, AAV7, AAV8, AAV9, AAV10, AAV11, AAV12 or AAV13, as well as any one of the more than 100 variants isolated from human and nonhuman primate tissues (See, e.g., Choi et al., 2005, Curr Gene Ther. 5: 299-310, 2005 and Gao et al, 2005, Curr Gene Ther. 5: 285-297). AAV vectors of any serotype may be used in the present invention, and the selection of AAV serotype will depend in part on the cell type(s) that were targeted for gene therapy. For treatment of WD, the liver is one of the relevant target organs. In some embodiments, the AAV vector is selected from serotype 9 (AAV9), serotype 8 (AAV8), serotype 5 (AAV5), or variant thereof. In an exemplary embodiment, the AAV vector is serotype 8 (AAV8) or a variant thereof.

In another aspect, this disclosure provides a recombinant adeno-associated virus (rAAV) comprising an AAV capsid the said AAV vector.

In some embodiment, the AAV capsid is from an AAV of serotype 9, 8, 1, 2, 3, 3B, 4, 5, 6, 7, 10, 11, 12, 13, rh10, or hu37 as well as any one of the AAV serotype isolated from human and nonhuman mammalians or variant thereof.

In some embodiment, the AAV is selected from the group consisting of: AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LKO4, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAV-PHP.B (PHP.B), AAV-PHP.A (PHP.A), G2B-26, G2B-13, TH1.1-32, TH1.1-35, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3, AAVG2B4, AAVG2B5 and variants thereof.

### Pharmaceutical Compositions:

In some embodiments, the rAAV is formulated in a buffer/carrier suitable for infusion in human subjects. The buffer/carrier should include a component that prevents the rAAV from sorption to the infusion tubing but does not interfere with the rAAV biopotency and transduction activity in vivo.

### Method for Treating a Disease

In another aspect, this disclosure provides a method for treating a disease in the subjects, comprising administration the effective amount of the said truncated ATP7B, the said nucleic acid sequence, the said recombinant nucleic construct, the said recombinant vector, the said rAAV, or the said pharmaceutical composition.

In some embodiment, the disease is an ATP7B related disease, preferably is Wilson's disease.

In some embodiment, the subject is mammalian, preferably human.

### EXAMPLES

Unless otherwise specified, the following general methods were followed in the examples described below.

rAAV Production

AAV8 or AAV9 particles were produced by transient triple-transfection of HEK293T cells or suspension HEK293 cells with plasmids encoding the AAV Rep, AAV Cap, and adenoviral helper genes, as well the recombinant genome containing the ATP7B construct. rAAV particles were purified using iodixanol-based density gradient ultracentrifugation. rAAV was then quantified by probe-based droplet digital PCR (ddPCR, Bio-Rad) assay and characterized by silver staining.

r-AAV Biopotency Assay

rAAV biopotency assay was performed by cell transduction using Huh-7 cell lines. Cells were plated in 15 cm² plate at a cell density of 8.5×10⁶ cells per plate. 24 hours later, cells were transfected with pGL4×MRE-LUC, as well as the pCMV-RL plasmid as internal control. After another 48 hours, cells were trypinized and seeded in 96-well plates at a cell density of 1.5×10′ cells/well 24 hours before transduction. rAAV transduction was performed at defined multiplicity of infection (MOI) of 3×10⁶. 48 hours after transduction, 150 μM CuSO₄ was supplemented for 24 hours before collection. Cells without any copper supplementary were collected for baseline measurement, and the relative light units were calculated by normalizing Firefly luciferase activity with Renilla luciferase activity.

### Dual Luciferase Assay

A reporter plasmid (pGL4×MRE-LUC) was used to assess the functional copper transportation capacity of the truncated ATP7B transgenes cloned in the pCMV vectors. This reporter plasmid expressed Firefly luciferase reporter gene under the transcriptional control of the metal response element (MRE), which responds to bioavailable cytosolic copper. HEK293T cells were co-transfected with pGL4×MRE-LUC and a plasmid expressing truncated ATP7B under the control of the CMV promoter, or the empty pcDNA 3.1 plasmid as a control. All cells were co-transfected with the pCMV-RL plasmid for internal control. Cells were incubated with 75 μM CuSO₄ for 24 h while the absence of copper overload is considered as the basal state. Firefly luciferase activity is normalized with Renilla luciferase activity to calculate the relative light units.

### Mouse Model Study Design

AAV vector containing the truncated ATP7B transgene were administered through tail vein injection of ATP7B^{-/-} mice at age of 8-12 weeks old. All mice were maintained under a special pathogen-free environment and in individually ventilated cages followed with institutional IACUC protocols. All cages, cob bedding, and water were sterilized before use. The cob bedding, food and water were changed twice a week.

AAV was dosed at 2.0×10¹² vg/kg. To assess the kinetics and durability of transgene expression, serum ceruloplasmin activity, alanine aminotransferase level and metabolic copper content were measured at various time points intervals post injection. Mice were followed up to the endpoint of study and sacrificed for tissue copper accumulation, biochemical and pathological analysis.

### Serum Metabolite and Tissue Collection

Serum was separated from fresh blood standing for 4 hour at 4° C. without anticoagulation by centrifugation at 12,000 rpm for 15 mins. Serum was stored at −80° C. Metabolic cages were used to collect 24-hour urine and feces for metabolic copper content.

Mice were anesthetized and euthanized, the tissues were collected from the mice after perfusion with saline, snap frozen and stored at −80° C. Tissue samples were divided into 4 parts, with 3 parts frozen separately in individual tubes and stored at −80° C. for tissue copper accumulation analysis, vector genome copy number and mRNA analysis. The remaining part was fixed with 10% neutral buffered formalin solution (NBF, pH 7.4) for about 24-48 hours at 4° C. for further histology analysis.

### Ceruloplasmin Activity, Alanine Aminotransferase Level, Metabolic Copper Content and Tissue Copper Accumulation

Serum, metabolite and tissue of mice were collected as described above.

Ceruloplasmin activity assay kit (Nanjing Jiancheng Bioengineering Institute, China) with o-dianisidine dihydrochloride as substrate (Stepien and Guy 2018) was used to detect ceruloplasmin activity in serum according to the manufacturer's instructions with modifications. Briefly, 5 μL diluted serum was added into 96-well plates, followed by adding 80 μL reagent 1 and subsequent 20 μL reagent 11. Samples were mixed well and incubated at 37° C. for 140 mins. A 20-min incubation was synchronously carried out for baseline detection and calculation. Finally, 200 μL reagent III were added after incubation for discontinuation and the absorbance was measured spectrophotometrically (Varioskan™ LUX, ThermoFisher) at 540 nm with ddH₂O as a blank.

The alanine aminotransferase assay kit (Nanjing Jiancheng Bioengineering Institute, China) was used to detect ALT activity in serum according to the manufacturer's instructions with modifications. Briefly, 10 μL diluted serum was pipetted into 96-well plates, followed by adding 200 μL reagent R1, and then were incubated for 10 mins at 37° C. Finally, 50 μL reagent R2 was added and mixed well. Absorbance was measured spectrophotometrically (Varioskan™ LUX, ThermoFisher) at 340 nm every 2 minutes, ten times, with ddH₂O as a blank.

Solid samples (feces or tissues) were weighed, homogenized and followed by drying to constant weight. Samples were then digested in the nitric acid solution for overnight. Urine samples were first centrifuged to remove insoluble or suspension particles. Appropriate dilution of solid samples with 10% nitric acid and 1% hydrogen peroxide, and of urine samples with 1% nitric acid and 0.5% hydrochloric acid was carried out before analysis. Samples were then assayed for copper content by Inductively coupled plasma mass spectrometry (ICP-MS), accomplishing by direct calibration using working aqueous standards.

### Vector Genome Copy Number, Relative RNA Transcription Level

To determine the vector genomic copy number in tissue samples post-rAAV injection, DNA was isolated from frozen liver samples by DNeasy Blood and Tissue Kit (QIAGEN) following manufacturer's instructions. After DNA isolation, probe-based qPCR (Roche) was performed to determine the vector genome copy number/reaction. Cell number/reaction was calculated according to the DNA amount quantification results. Vector genome copy number/cell was then calculated by the normalization of genome copy/reaction to cell number/reaction.

To determine the relative RNA transcription level in tissue samples post-rAAV injection, RNA was isolated from frozen liver samples using RNeasy Kit (QIAGEN) following manufacturer's instructions. After RNA isolation, cDNA was synthesized using PrimeScript RT Master Mix (TAKARA). 500 ng RNA was applied to each reverse transcription reaction. cDNA was then diluted and been applied to probe-based qPCR (Roche) test with mouse Gapdhas housekeeping gene.

### Immunohistochemistry

Rabbit anti-human ATP7B antibody (1:100, ThermoFisher, PA5-102826) was used to visualize cells expression the said truncated ATP7B in mice. The formalin-fixed mouse tissues were deparaffined with xylene and graded ethanol ashes, followed by antigen retrieval using pepsin according to manufacturer's recommendations. Sections were counterstained with hematoxylin. Then HRP labeled secondary antibody was used for detecting. The signals were visualized by using Pierce™ Western Blot Signal Enhancer (Thermo Scientific) according to the manufacturer's instructions.

### Statistical Analysis

Statistical analysis was performed using Prism 8 (GraphPad) software. Columns analysis was performed by one-way ANOVA. P-values and sample size were indicated in figure descriptions.

### Example 1: Contribution of Separate CREs of ATP7B to Copper Transportation Capacity

ATP7B, a multi-domain protein, was composed of several independently folded copper (II) responsive elements (CREs) between the leader peptide (LP) and C-terminal region (CTR), of which sequences were represented in Table 2. According to the protein structure, six CREs and five endogenous linkers connecting CREs could be identified from the N-terminal to the membrane-spanning part of ATP7B, which contains four beta strands and two helixes in each CRE.

TABLE 2
Sequences of domains composing ATP7B

| | | SEQ | | SEQ |
|---|---|---|---|---|
| Name | Note | ID NO | Note | ID NO |
| CRE1 | 190-376 bp of | 10 | 64-125 AA of | 1 |
| | SEQ ID NO. 35 | | SEQ ID NO. 36 | |
| CRE2 | 430-630 bp of | 11 | 144-210 AA of | 2 |
| | SEQ ID NO. 35 | | SEQ ID NO. 36 | |
| CRE3 | 772-981 bp of | 12 | 258-327 AA of | 3 |
| | SEQ ID NO. 35 | | SEQ ID NO. 36 | |
| CRE4 | 1078-1278 bp of | 13 | 360-426 AA of | 4 |
| | SEQ ID NO. 35 | | SEQ ID NO. 36 | |
| CRE5 | 1465-1665 bp of | 14 | 489-555 AA of | 5 |
| | SEQ ID NO. 35 | | SEQ ID NO. 36 | |
| CRE6 | 1693-1893 bp of | 15 | 565-631 AA of | 6 |
| | SEQ ID NO. 35 | | SEQ ID NO. 36 | |
| Leader | 1-189 bp of | 9 | 1-63 AA of | 7 |
| peptide | SEQ ID NO. 35 | | SEQ ID NO. 36 | |
| (LP) | | | | |
| C-termial | 1894-4395 bp of | 16 | 632-1465 AA of | 8 |
| region | SEQ ID NO. 35 | | SEQ ID NO. 36 | |
| (CTR) | | | | |
| Linker1 | 376-429 bp of | 42 | 126-143 AA of | 37 |
| | SEQ ID NO. 35 | | SEQ ID NO. 36 | |
| Linker2 | 631-771 bp of | 43 | 211-257 AA of | 38 |
| | SEQ ID NO. 35 | | SEQ ID NO. 36 | |
| Linker3 | 982-1077 bp of | 44 | 328-359 AA of | 39 |
| | SEQ ID NO. 35 | | SEQ ID NO. 36 | |
| Linker4 | 1279-1464 bp of | 45 | 427-488 AA of | 40 |
| | SEQ ID NO. 35 | | SEQ ID NO. 36 | |
| Linker5 | 1666-1692 bp of | 46 | 556-564 AA of | 41 |
| | SEQ ID NO. 35 | | SEQ ID NO. 36 | |

Here, every single GRE was cloned into pCMV vectors respectively with the LP and CTR as truncated ATP7B transgenes, as described in Table 3, to determine the individual contribution of single GRE to the copper transportation capacity via Dual Luciferase Assay in HEK293T cells. Higher relative Fluci/Rluci represented lower copper transportation capacity while the absence of copper overload was considered as the basal state. The LYM3P016 vector encoding wild type complete ATP7B was used as positive control. The results demonstrated that LYM3P027-CRE5 exhibited the highest copper transportation capacity among six CREs, indicating that CRE5 majorly contributes to the copper transportation capacity of ATP7B3 (FIG. 1).

TABLE 3
Description of the constitution of vectors with single CRE

| | Description of |
|---|---|
| Vectors | the constitution |
| LYM3P016 | Complete ATP7B |
| LYM3P132 | LP + CRE1 + CTR |
| LYM3P133 | LP + CRE2 + CTR |
| LYM3P134 | LP + CRE3 + CTR |
| LYM3P135 | LP + CRE4 + CTR |
| LYM3P027 | LP + CRE5 + CTR |
| LYM3P026 | LP + CRE6 + CTR |

### Example 2: Copper Export Capacity of CRE Scheme Based on CRE5

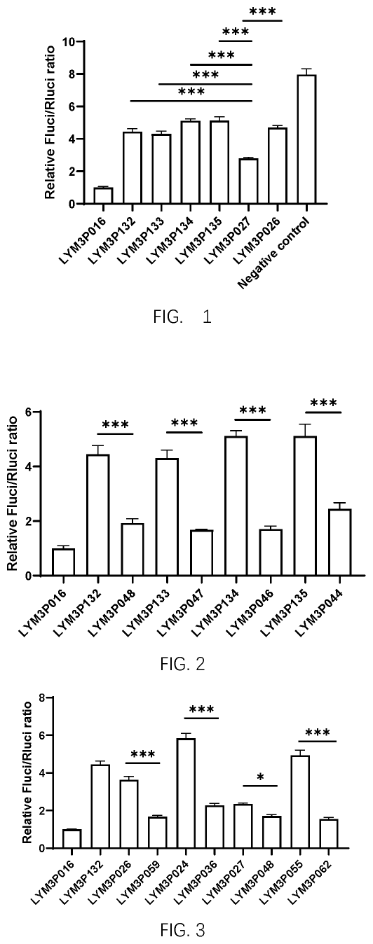

As proved above, CRE5 had been demonstrated to majorly contribute to the copper transportation capacity of ATP7B. To compensate the reduced copper transportation capacity of truncated ATP7 with CRE1, i0 CRE2, CRE3 or CRE4 alone, GRE combination scheme based on CRE5 were built and cloned into the pCMV vector respectively as described in Table 4, inwhich CREswerejoined throughthe endogenouslinkersconnecting CREs in ATP7B. The LYM3P316 vector encoding wild type complete ATP7B was used as positive control. The copper transportation capacity of the truncated ATP7B were evaluated via Dual Luciferase Assay in HEK293T cells, in which higher relative Fluci/Rluci represents lower copper transportation capacity. As shown in FIG. 2, all constructs with the combination of CRE5 dramatically increased copper transportation capacity of other CREs that without CRE5, which further indicated the significant contribution of CRE5 to the copper transportation capacity of ATP7B.

TABLE 4
Description of the vector constitution with CRE5 combination

| | Description of |
|---|---|
| Vectors | the constitution |
| LYM3P016 | Complete ATP7B |
| LYM3P132 | LP + CRE1 + CTR |
| LYM3P048 | LP + CRE1 + Linker4 + CRE5 + CTR |
| LYM3P133 | LP + CRE2 + CTR |
| LYM3P047 | LP + CRE2 + Linker4 + CRE5 + CTR |
| LYM3P133 | LP + CRE3 + CTR |
| LYM3P046 | LP + CRE3 + Linker4 + CRE5 + CTR |
| LYM3P134 | LP + CRE4 + CTR |
| LYM3P044 | LP + CRE4 + Linker4 + CRE5 + CTR |

### Example 3: Combination of CRE1 or CRE2 to Other CREs Increased Copper Transportation Capacity Significantly

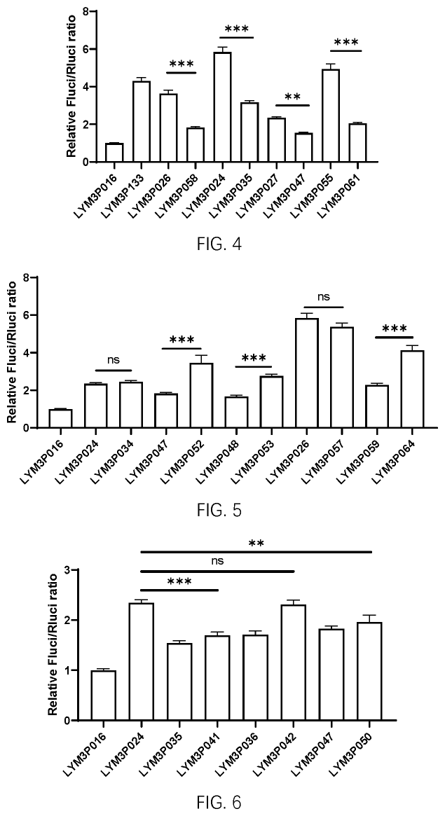

To explore the additive effect of CRE1, CRE2 or CRE3 on the copper transportation capacity of other CREs or CRE-schemes, truncated ATP7B transgenes with the combination of CRE1, CRE2 or CRE3 with other CRE or CRE-schemes were cloned into the pCMV vectors as listed in Tables 5-7, in which CREs were joined through the endogenous linkers connecting CREs in ATP7B. The LYM3P016 vector encoding wild type complete ATP7B was used as positive control. The copper transportation capacity of vectors listed in Tables 5-7 that encode the truncated ATP7B were evaluated via Dual Luciferase Assay in HEK293T cells. Addition of CRE1 or CRE2 to other CREs or CRE-schemes as the constructs of LYM3P059, LYM3P036, LYM3P048, LYM3P062, LYM3P058, LYM3P035, LYM3P047, and LYM3P061, were all found to significantly improve the copper transportation capacity of the constructs of LYM3P026, LYM3P024, LYM3P027, and LYM3P055 respectively which encode ATP7B without CRE1 or CRE2 (FIG. 3, FIG. 4). In the contrary, adding CRE3 to other CREs or CRE-schemes, as the constructs of LYM3P034, LYM3P052, LYM3P053, LYM3P057, and LYM3P064, had no further enhancement effects, while some even had negative effects, on the copper transportation capacity of constructs of LYM3P024, LYM3P047, LYM3P048, LYM3P026, and LYM3P059 respectively which encode ATP7B without CRE3 (FIG. 5).

The constructs of LYM3P041, LYM3P042 and LYM3P050 were cloned by further combination of CRE3 or CRE4 on the constructs of LYM3P035, LYM3P036 and LYM3P047, the result showed that addition of CRE3 or CRE4 had slight negative effect on the copper transportation capacity on LYM3P035, LYM3P036 and LYM3P047. However, constructs of LYM3P041, LYM3P042 and LYM3P050 that encode CRE combinations with CRE3 or CRE4 still overweighed or be comparable to the constructs of LYM3P024 that encode CRE5+CRE6 in the copper transportation capacity (FIG. 6).

TABLE 5
Description of the vector constitution with CRE1 combinations

| | Description of |
|---|---|
| Vectors | the constitution |
| LYM3P016 | Complete ATP7B |
| LYM3P132 | LP + CRE1 + CTR |
| LYM3P027 | LP + CRE5 + CTR |
| LYM3P048 | LP + CRE1 + Linker4 + CRE5 + CTR |
| LYM3P026 | LP + CRE6 + CTR |
| LYM3P059 | LP + CRE1 + Linker5 + CRE6 + CTR |
| LYM3P024 | LP + CRE5 + Linker5 + CRE6 + CTR |
| LYM3P036 | LP + CRE1 + Linker4 + CRE5 + Linker5 + CRE6 + CTR |
| LYM3P055 | LP + CRE4 + Linker5 + CRE6 + CTR |
| LYM3P062 | LP + CRE1 + Linker3 + CRE4 + Linker5 + CRE6 + CTR |
| LYM3P051 | LP + CRE1 + Linker3 + CRE4 + Linker4 + CRE5 + CTR |

TABLE 6
Description of the vector constitution with CRE2 combinations

| | Description of |
|---|---|
| Vectors | the constitution |
| LYM3P016 | Complete ATP7B |
| LYM3P133 | LP + CRE2 + CTR |
| LYM3P027 | LP + CRE5 + CTR |
| LYM3P047 | LP + CRE2 + Linker4 + CRE5 + CTR |
| LYM3P026 | LP + CRE6 + CTR |
| LYM3P058 | LP + CRE2 + Linker5 + CRE6 + CTR |
| LYM3P024 | LP + CRE5 + Linker5 + CRE6 + CTR |
| LYM3P035 | LP + CRE2 + Linker4 + CRE5 + Linker5 + CRE6 + CTR |
| LYM3P055 | LP + CRE4 + Linker5 + CRE6 + CTR |
| LYM3P061 | LP + CRE2 + Linker3 + CRE4 + Linker5 + CRE6 + CTR |
| LYM3P050 | LP + CRE2 + Linker3 + CRE4 + Linker4 + CRE5 + CTR |

TABLE 7
Description of the vector constitution
with CRE3 or CRE4 combinations

| | Description of |
|---|---|
| Vectors | the constitution |
| LYM3P016 | Complete ATP7B |
| LYM3P024 | LP + CRE5 + Linker5 + CRE6 + CTR |
| LYM3P034 | LP + CRE3 + Linker4 + CRE5 + Linker5 + CRE6 + CTR |
| LYM3P047 | LP + CRE2 + Linker4 + CRE5 + CTR |
| LYM3P052 | LP + CRE2 + Linker2 + CRE3 + Linker4 + CRE5 + CTR |
| LYM3P048 | LP + CRE1 + Linker4 + CRE5 + CTR |
| LYM3P053 | LP + CRE1 + Linker2 + CRE3 + Linker4 + CRE5 + CTR |
| LYM3P026 | LP + CRE6 + CTR |
| LYM3P057 | LP + CRE3 + Linker5 + CRE6 + CTR |
| LYM3P059 | LP + CRE1 + Linker5 + CRE6 + CTR |
| LYM3P064 | LP + CRE1 + Linker2 + CRE3 + Linker5 + CRE6 + CTR |
| LYM3P035 | LP + CRE2 + Linker4 + CRE5 + Linker5 + CRE6 + CTR |
| LYM3P041 | LP + CRE2 + Linker2 + CRE3 + Linker4 + CRE5 + |
| | Linker5 + CRE6 + CTR |
| LYM3P036 | LP + CRE1 + Linker4 + CRE5 + Linker5 + CRE6 + CTR |
| LYM3P042 | LP + CRE1 + Linker2 + CRE3 + Linker4 + CRE5 + |
| | Linker5 + CRE6 + CTR |
| LYM3P050 | LP + CRE2 + Linker3 + CRE4 + Linker4 + CRE5 + CTR |

### Example 4: Combination of CRE1+CRE2 to Other CREs or CRE-Schemes Significantly Increased Copper Transportation Capacity

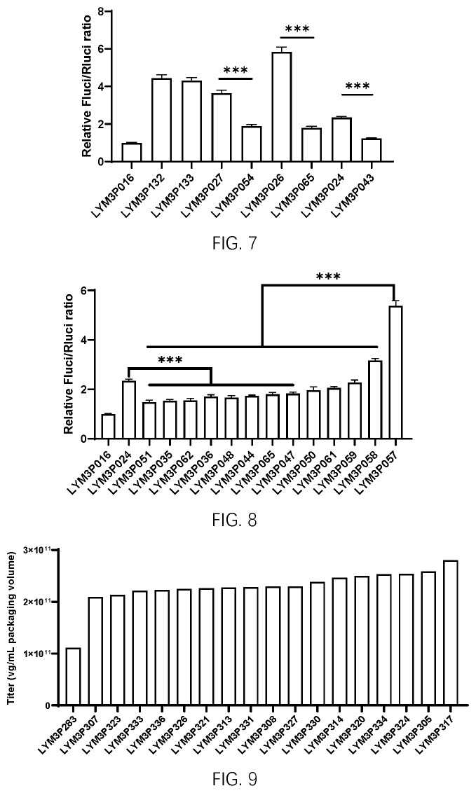

While CRE1 and CRE2 could increase coppertransportation capacity independently when combining with other GREs or GRE-schemes, the effect of CRE1+CRE2 remains questionable. To evaluate the additive effect of CRE1+CRE2 on copper transportation capacity of other GRE or GRE-schemes, truncated ATP7B transgenes with or without CRE1+CRE2 were cloned into the pGMV vectors as described in Table 8, in which GREs were joined through the endogenous linkers connecting GREs in ATP7B3. The copper transportation capacity of the truncated ATP7B transgenes were evaluated via Dual Luciferase Assay in HEK293T cells while the LYM3P016 vector encoding wild type complete ATP7B3 was used as positive control. The constructs of LYM3P054, LYM3P065, and LYM3P043 with the scheme of CRE1+CRE2 all showed increased copper transportation capacity compared to the constructs of LYM3P027, LYM3P026, and LYM3P024, respectively that encoded only CRE5, CRE6 or CRE5+CRE6, indicating that addition of CRE1+CRE2 could significantly improve the copper transportation capacity (FIG. 7).

TABLE 8
Description of the vector constitution
with CRE1 + CRE2 combination

| | Description of |
|---|---|
| Vectors | the constitution |
| LYM3P016 | Complete ATP7B |
| LYM3P132 | LP + CRE1 + CTR |
| LYM3P133 | LP + CRE2 + CTR |
| LYM3P027 | LP + CRE5 + CTR |
| LYM3P054 | LP + CRE1 + Linker1 + CRE2 + Linker4 + CRE5 + CTR |
| LYM3P026 | LP + CRE6 + CTR |
| LYM3P065 | LP + CRE1 + Linker1 + CRE2 + Linker5 + CRE6 + CTR |
| LYM3P024 | LP + CRE5 + Linker5 + CRE6 + CTR |
| LYM3P043 | LP + CRE1 + Linker1 + CRE2 + Linker4 + CRE5 + |
| | Linker5 + CRE6 + CTR |

### Example 5: CRE Combinations Containing Specific CREs Exhibit Comparative Copper Transportation Capacity to Wild Type Form

Full-length ATP7B transgene, which is about 4.4 kb, could be oversized for packaging into adeno-associated virus (AAV). Therefore, it was necessary to obtain a truncated form with comparative copper transportation capacity to wild type ATP7B for developing treatments with gene therapy vectors against Wilson's disease. Considering AAV packaging limitation, three or less CRE combinations were preferable. Truncated ATP7B transgenes with three or less CRE combinations have been cloned into the pCMV vectors. The copper transportation capacity of the above constructs was evaluated via Dual Luciferase Assays in HEK293T cells. As shown in the results, these constructs with CRE combinations of LYM3P051, LYM3P035, LYM3P062, LYM3P036, LYM3P048, LYM3P044, LYM3P065 and LYM3P047 were found to exhibit comparative copper transportation capacity to LYM3P016 that encode wild type form of ATP7B. Meanwhile, the constructs of LYM3P051, LYM3P035, LYM3P062, LYM3P036, LYM3P048, LYM3P044, LYM3P065 and LYM3P047 were shown to have significant higher copper transportation capacity than the constructs of LYM3P024 that encode CRE5+CRE6. The constructs of LYM3P051, LYM3P035, LYM3P062, LYM3P036, LYM3P048, LYM3P044, LYM3P065, LYM3P047, LYM3P050, LYM3P061, LYM3P059 and LYM3P058 were all shown to have significant higher copper transportation capacity than the constructs of LYM3P057 which contains CRE3+CRE6 (FIG. 8).

### Example 6: Truncated ATP7B Transgenes with Specific Combinations of CRE or CREs could be Produced Efficiently in AAV and Showed Therapeutic Effects Against Wilson's Disease in Mice Model

To verify whether the truncated ATP7B transgenes, which had outstanding copper transportation capacity described above, were suitable for AAV production with considerable yield, truncated ATP7B transgenes with specific combinations of CRE or CRE-schemes were cloned into the AAV vectors under the control of a liver-specific promoter as described in Table 9. The production of viral particles was performed as described above in Example 1. The yield of packaged AAV was then evaluated via ddPCR with specific primers and probe sets. As shown in FIG. 9, all constructs with truncated ATP7B transgenes with three or less CREs could be successfully packaged into AAV8 with the average yield more than 2.0×10¹¹ vg/mL in suspension HEK293 cells system, which is 2-fold higher than the LYM3P283 with complete ATP7B transgene.

TABLE 9
Description of the AAV vector constitutions
with different CREs combinations

| | Description of |
|---|---|
| Vectors | the constitutions |
| LYM3P283 | Complete ATP7B |
| LYM3P305 | LP + CRE5 + Linker5 + CRE6 + CTR |
| LYM3P307 | LP + CRE6 + CTR |
| LYM3P308 | LP + CRE5 + CTR |
| LYM3P313 | LP + CRE2 + Linker4 + CRE5 + Linker5 + CRE6 + CTR |
| LYM3P314 | LP + CRE1 + Linker4 + CRE5 + Linker5 + CRE6 + CTR |
| LYM3P317 | LP + CRE4 + Linker4 + CRE5 + CTR |
| LYM3P320 | LP + CRE2 + Linker4 + CRE5 + CTR |
| LYM3P321 | LP + CRE1 + Linker4 + CRE5 + CTR |
| LYM3P323 | LP + CRE2 + Linker3 + CRE4 + Linker4 + CRE5 + CTR |
| LYM3P324 | LP + CRE1 + Linker3 + CRE4 + Linker4 + CRE5 + CTR |
| LYM3P326 | LP + CRE1 + Linker1 + CRE2 + Linker4 + CRE5 + CTR |
| LYM3P327 | LP + CRE4 + Linker5 + CRE6 + CTR |
| LYM3P330 | LP + CRE2 + Linker5 + CRE6 + CTR |
| LYM3P331 | LP + CRE1 + Linker5 + CRE6 + CTR |
| LYM3P333 | LP + CRE2 + Linker3 + CRE4 + Linker5 + CRE6 + CTR |
| LYM3P334 | LP + CRE1 + Linker3 + CRE4 + Linker5 + CRE6 + CTR |
| LYM3P336 | LP + CRE1 + Linker1 + CRE2 + Linker5 + CRE6 + CTR |

In vivo therapeutic effects of above successfully produced AAVs were then evaluated in Atp7b^{-/-} mice that had been built as described in Example 1. Briefly, 12-week Atp7b^{-/-} mice and naïve mice were administrated with 2.0×10¹² vg/kg AAV or vehicle only through tail vein injection. After AAV administration, serums were collected periodically to evaluate the ceruloplasmin (Cp) activity. 4 weeks later, mice were sacrificed to determine the tissue copper accumulation by both histology and ICP-MS.

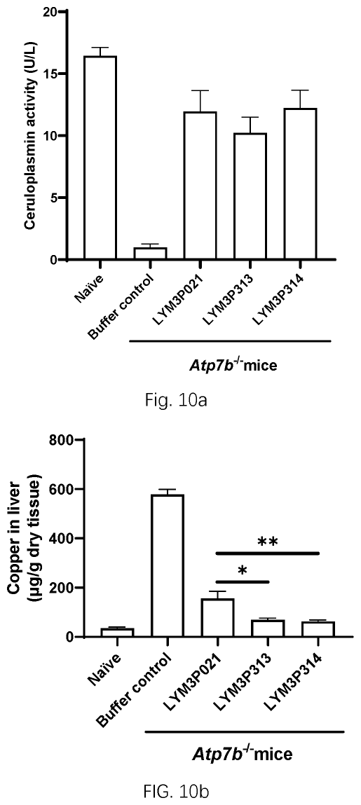

It was found that MAVs carrying truncated ATP7B transgenes with specific GRE-schemes of CRE2+CRE5+CRE6 and CRE1+CRE5+CRE6, which are LYM3P313 and LYM3P314 respectively, could reverse ceruloplasmin activity in Wilson's disease mice model, which equivalent to that of LYM3P021 that encode CRE-scheme of CRE5+CRE6 (FIG. 10a). Moreover, the results showed that LYM3P313 and LYM3P314 decreased the copper accumulation in liver with significantly higher efficiency than that of LYM3P021 (FIG. 10b).

1. A truncated ATP7B, comprising one or more sequences selected from CRE5, CRE6, and variants thereof, and one or more sequences selected from CRE1, CRE2, and variants thereof.

2. The truncated ATP7B of claim 1, optionally comprising a flexible linker connecting the sequences to each other.

3. The truncated ATP7B of claim 1, having a structure as shown in Formula (Ia) from N-terminus to C-terminus:
A-L-B-L-C-L-D (Ia);
wherein A, B, C, or D is selected from CRE1, CRE2, CRE5, CRE6 and variants thereof; A, B, C, and D are different, respectively; A and B are present, C and/or D are optionally present; and
L is none or a flexible linker.

4. The truncated ATP7B of claim 1, wherein 1) CRE5 or variant thereof and/or 2) CRE6 or variant thereof is present; and 3) CRE1 or variant thereof and/or 4) CRE2 or variant thereof is present.

5. The truncated ATP7B of claim 1, wherein CRE1 or variant thereof, CRE5 or variant thereof, and CRE6 or variant thereof are present;
wherein CRE2 or variant thereof, CRE5 or variant thereof, and CRE6 or variant thereof are present;
wherein CRE1 or variant thereof, CRE2 or variant thereof, and CRE5 or variant thereof are present;
wherein CRE1 or variant thereof, CRE2 or variant thereof, and CRE6 or variant thereof are present;
wherein CRE1 or variant thereof, CRE2 or variant thereof, CRE5 or variant thereof, and CRE6 or variant thereof are present; wherein CRE1 or variant thereof and CRE5 or variant thereof are present; wherein CRE1 or variant thereof and CRE6 or variant thereof are present; wherein CRE2 or variant thereof and CRE5 or variant thereof are present; or
wherein CRE2 or variant thereof and CRE6 or variant thereof are present.

6.-13. (canceled)

14. The truncated ATP7B of claim 1, wherein the truncated ATP7B comprises an amino acid sequence selected from the group consisting of SEQ ID NOs: 22, 28, 30, 32, 34, 18, 20, 24, and 26, and an amino acid sequence that is at least 80%, at least 85%, at least 90%, at least 95%, at least 99%, at least 99.5%, or at least 99.8% identical to any one of SEQ ID NOs: 22, 28, 30 32, 34, 18, 20, 24, and 26,
wherein the truncated ATP7B retains the functionality of transmembrane copper transportation.

15. The truncated ATP7B of claim 4, wherein CRE3 is deleted;
wherein CRE4 or variant thereof is present; or
wherein CRE3 or variant thereof is present.

16.-17. (canceled)

18. A nucleic acid sequence encoding the truncated ATP7B of claim 1.

19. The nucleic acid sequence of claim 18, wherein the nucleic acid sequence comprises a sequence selected from the group consisting of SEQ ID NOs: 21, 27, 29, 31, 33, 17, 19, 23, and 25, and a sequence that is at least 80%, at least 85%, at least 90%, at least 95%, at least 99%, at least 99.5%, or at least 99.8% identical to any one of SEQ ID NOs: 21, 27, 29, 31, 33, 17, 19, 23, and 25, wherein the truncated ATP7B retains the functionality of transmembrane copper transportation.

20. A RNA polynucleotide encoding the truncated ATP7B of claim 1.

21. The RNA polynucleotide of claim 20, wherein the RNA polynucleotide comprises a messenger RNA (mRNA) or a circular RNA (circRNA).

22. A recombinant nucleic acid construct comprising:
(a) a 5′-inverted terminal repeat (ITR) sequence;
(b) a promoter sequence;
(c) the nucleic acid sequence of claim 18;
(d) a poly(A) sequence; and
(e) a 3′-ITR sequence.

23. The recombinant nucleic acid construct of claim 22, wherein the recombinant nucleic acid construct further comprises one or more enhancer sequences, and/or one or more intron sequences.

24. (canceled)

25. A vector comprising the nucleic acid sequence of claim 18.

26. The vector of claim 25, wherein the vector is a virus vector; preferably an AAV vector.

27. (canceled)

28. A recombinant adeno-associated virus (rAAV) comprising the vector of claim 26.

29. The rAAV of claim 28, wherein the AAV is selected from the group consisting of: serotype 9, 8, 1, 2, 3, 3B, 4, 5, 6, 7, 10, 11, 12, 13, rh10, or hu37 as well as any one of the AAV serotype isolated from human and nonhuman mammalians or variant thereof.

30. A pharmaceutical composition comprising the truncated ATP7B of claim 1, and a pharmaceutically acceptable carrier.

31. A method for treating a disease in a subject, comprising administrating an effect amount of the truncated ATP7B of claim 1.

32. The method of claim 31, wherein the disease is an ATP7B related disease, or
Wilson's disease; and/or
wherein the subject is mammalian, preferably human.

33.-34. (canceled)
